(19) United States
(12) Patent Application Publication
(10) Pub. No.: US 2022/0308060 A1
(43) Pub. Date: Sep. 29, 2022

(54) THERAPY FOR HEART DISORDERS

(71) Applicant: The Francis Crick Institute Limited, London (GB)

(72) Inventor: Andreia Bernardo, London (GB)

(21) Appl. No.: 17/616,883

(22) PCT Filed: Jun. 5, 2020
(86) PCT No.: PCT/GB2020/051381

§ 371 Date: Dec. 6, 2021

(30) Foreign Application Priority Data
Jun. 7, 2019 (GB) 1908180.1

(51) Int. Cl. G01N 33/574; C12N 5/077; A61P 9/04

(52) U.S. Cl. CPC G01N 33/57484; C12N 5/0657; A61P 9/04

(57) ABSTRACT

The present invention relates to a population of cells with an increased proportion of left ventricular cardiomyocytes, and uses thereof, for example in treatment of disorders of the left ventricle and use in screening for drugs which may be used to treat disorders of the left ventricle.

### FIELD OF THE INVENTION

The present invention relates to a population of cells comprising an increased proportion of left ventricular cardiomyocytes, and uses thereof, for example in treatment of disorders of the left ventricle and use in screening for drugs which may be used to treat disorders of the left ventricle.

### BACKGROUND

Cardiovascular disease is the leading cause of death in industrialized countries. Myocardial infarction (MI), which leads to a dramatic loss of contractile heart muscle in the left ventricle (LV), is the most common cause of heart injury. If the infarct is big, or if patients suffer multiple infarcts, they often end up with heart failure for which the only effective treatment is a heart transplant. The life span of heart failure patients is up to 5 years, with as many as 60% dying within the first year from being diagnosed. Thus, safe and long-lasting treatments by which damaged heart muscle can be replaced upon MI/heart failure are urgently required. The key to improving the long-term outcome of patients is the repopulation of the LV by functional cardiomyocytes.

The present inventors have developed a new method for providing a population of cells, which results in a surprisingly high number or percentage of cardiomyocytes, which express markers specific for left ventricle cells. The method leads to rapid generation of left ventricular cardiomyocytes (20 days) that may exhibit a more mature identity than that which can be achieved using previous differentiation methods and long term cultures (60 days or over).

The present inventors therefore provide for the first time a population of cells which has a high level of homogeneity for left ventricle cardiomyocytes which may have a higher level of maturity, and a method for producing the same in an effective and rapid way. Such a population of cells may be used for a variety of purposes, for example as a cell therapy to treat disorders of the left ventricle, to model disorders of the left ventricle, or for drug screening to identify drugs which may be used to treat disorders of the left ventricle, or for cardiotoxicity tests.

### BRIEF DESCRIPTION OF THE INVENTION

The present invention provides a population of cells which comprises at least about 60% cells that are double-positive for the markers HAND1 and MLC2v. Such cells are left ventricular cardiomyocytes. The two markers HAND1 and MLC2v together are informative of left ventricular cardiomyocyte phenotype. This was not previously appreciated in the art.

The present invention allows efficient production of such a population of left ventricular cardiomyocytes for the first time. This provides a significant contribution to the art, for example for therapeutic use as discussed below. As described in the present Examples, evidence is provided that the cells according to the present invention are more mature at day 20 than cells achieved using monolayer differentiation methods known in the art, and long term cultures at 60 days. For example, immunofluorescence analysis for sarcomere markers is provided. Also provided is functional evidence from electrophysiology that the cells have a ventricular action potential shape (3 different types of assay were used). In addition, functional data from calcium imaging regarding the calcium responses are provided. We further provide evidence that these cells can successfully generate engineered heart tissues (EHTs). Lastly, we provide evidence that EHTs generated with left ventricle-like cardiomyocytes lose spontaneous pacemaker ability, which is in line with what is expected for more mature ventricular cardiomyocytes.

In one aspect the invention provides a method for treating a disorder of the left ventricle in a subject, comprising administering to said subject a population of cells which comprises at least about 60% cells that are double-positive for the markers HAND1 and MLC2v.

In one aspect the invention provides a population of cells which comprises at least about 60% cells that are double-positive for the markers HAND1 and MLC2v for use in the treatment or prevention of disorders of the left ventricle.

In one aspect the invention provides a population of cells which comprises at least about 60% cells that are double-positive for the markers HAND1 and MLC2v for use in the manufacture of a medicament for use in the treatment or prevention of a disorder of the left ventricle.

In one aspect the invention provides the use of a population of cells which comprises at least about 60% cells that are double-positive for the markers HAND1 and MLC2v for the treatment or prevention of a disorder of the left ventricle.

In one aspect the invention provides a method for preparing a population of cells which comprises at least about 60% cells that are double positive for the markers HAND1 and MLC2v, wherein said method comprises the step of culturing a pluripotent stem cell in a medium comprising a retinoic acid receptor antagonist or inverse agonist.

In one aspect said method may comprise a step of culturing said cell or cell population in a medium devoid of vitamin A, for example from day 8 of culture onwards.

In a further aspect the invention provides a method for screening for a drug suitable for treating or preventing a disorder of the left ventricle, wherein said method comprises contacting a population of cells as described herein with a candidate drug.

In yet a further aspect the invention provides a method for screening for cardiotoxicity in respect of an agent, wherein said method comprises contacting a population of cells as described herein with an agent.

### LIST OF FIGURES

FIG. 1 shows the retinoic acid pathway gene expression phenotype of the different chambers of the mouse heart. A Heat map showing the expression pattern per region and stage analysed. Samples were analysed from microdissected hearts as exemplified on the diagram on the right, where RV corresponds to the presumptive right ventricle, LV corresponds to the presumptive left ventricle and A corresponds to the presumptive atria chamber. The stages analysed (STO-ST3) correspond to a series of stages of heart development ranging from the early heart tube (E8.25) to the end of looping (E8.5) as depicted in the diagram on the right. Highlighted underneath are the genes which are consistently differentially expressed in a given region across all stages analysed, as indicated per the colour code. B Pathway enrichment overview as generated by the Reactome analysis tool when the genes uniquely upregulated in atrial samples were used as the query dataset. Scale colour bar denotes the p-value of enrichment. The arrow next to the scale bar denotes the p-value for the pathway: Retinoid metabolism and transport, and the enriched nodes for this pathway are highlighted in the corresponding p-value colour. Ci Reactome results for the pathway: Retinoid metabolism and transport, which ranked 16 out of the top 25 most relevant pathways found enriched within the gene-set constituted of genes upregulated uniquely in atrial samples. Cii List of genes found upregulated within the Retinoid metabolism and transport pathway as per the Reactome analysis of the genes upregulated uniquely in atrial samples. D Gorilla GO term analysis results for the Retinoic acid (RA) pathway related GO terms found enriched within the gene-set: genes upregulated uniquely in atrial samples. The FDR for the hypergeometric p-value is indicated in brackets. E Graphs showing the normalised read counts for RA direct target genes (i) or genes involved in the RA pathway (ii, as highlighted by the Reactome analysis in panel Cii). Expression is shown for the sample groups microdissected from the mouse hearts as highlighted in panel A, i.e. A (presumptive atrial), RV (presumptive right ventricle) and LV (presumptive left ventricle). * Denotes a significant difference (p<0.05 in red, p<0.001 in black) between the A and LV group and # denotes a significant difference (p<0.05 in red, p<0.001 in black) between the A both the LV and RV groups.

FIG. 2 shows hPSC growth and cardiac differentiation. A Schematic of the hPSC cardiomyocyte differentiation protocol indicating the different steps and components used in the protocol. B Representative images of hPSCs seeded as single cells for cardiac differentiation, followed by differentiation using the left ventricle cardiomyocyte protocol.

FIG. 3 shows chamber identity determination of cardiomyocytes produced using the left ventricle cardiomyocyte protocol. A Representative confocal micrographs showing immunostaining of MLC2V (green) in the top panel and HAND1 (green) in the bottom panel. Cells were stained on day 20 of the differentiation protocol. Cells were co-stained with TNNT2 to identify all cardiomyocytes and DAPI to visualise all cells. Scale bars represent 100 μm. B Representative flow cytometry analysis of the proportion of TNNT2⁺ cells in day 20 populations. Control unstained cells can be seen in blue. C Zoomed in confocal micrographs showing immunostaining of MLC2V (green) and HAND1 (red) to highlight the myofibril organisation at day 20 of the differentiation. Cells were co-stained with DAPI to visualise all cells. Scale bars represent 35 μm. D Representative flow cytometry analysis of the proportion of HAND1⁺/MLC2V⁺ cells in day 20 populations. E Graphs showing the relative expression levels of TBX5, IRX4 and MYL2 on days 0 (D0), 8 (D8), and 20 (D20) as determined by qRT-PCR. Data was normalised to the house keeping gene PBGD. T-tests were performed; * denotes a significant difference (p<0.01), and # denotes a significant difference (p<0.050).

FIG. 4 shows the functional characterization of cardiomyocytes produced using the left ventricle cardiomyocyte protocol. A LEAP signal for typical day 20 cardiomyocytes as acquired by the Axion Biosystems MEA system, demonstrating that at the individual cell level cells exhibit a ventricular action potential shape, i.e. a plateau followed by a sharp repolarization not seen in commercially available cardiomyocytes (grey line). Bi Field potential of an entire well of day 35 differentiated cardiomyocytes as measured by the Maxwell Biosystems MEA system containing 20000 electrodes. Bii Principal component analysis of the field potentials denoted in Bi showing that only one population of cells could be identified. C Graph showing the average beat rate of day 20 cardiomyocytes as determined using di-4-ANEPPS and optical mapping (CellOptic), demonstrating that even as early as day 20 of differentiation the beat rate for these cardiomyocytes is slow. The beat rate of commercially available cardiomyocytes is denoted by the grey line.

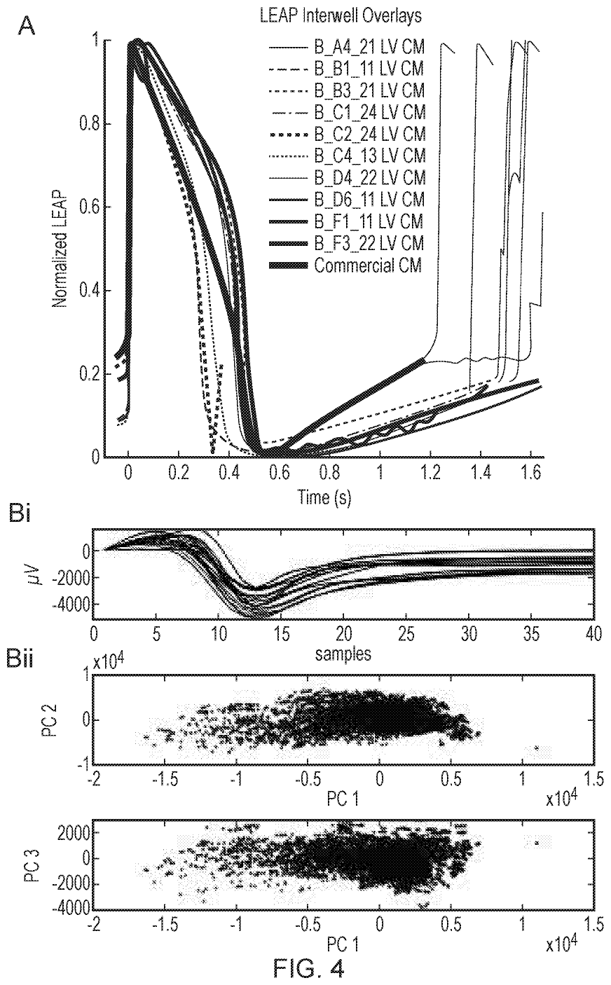

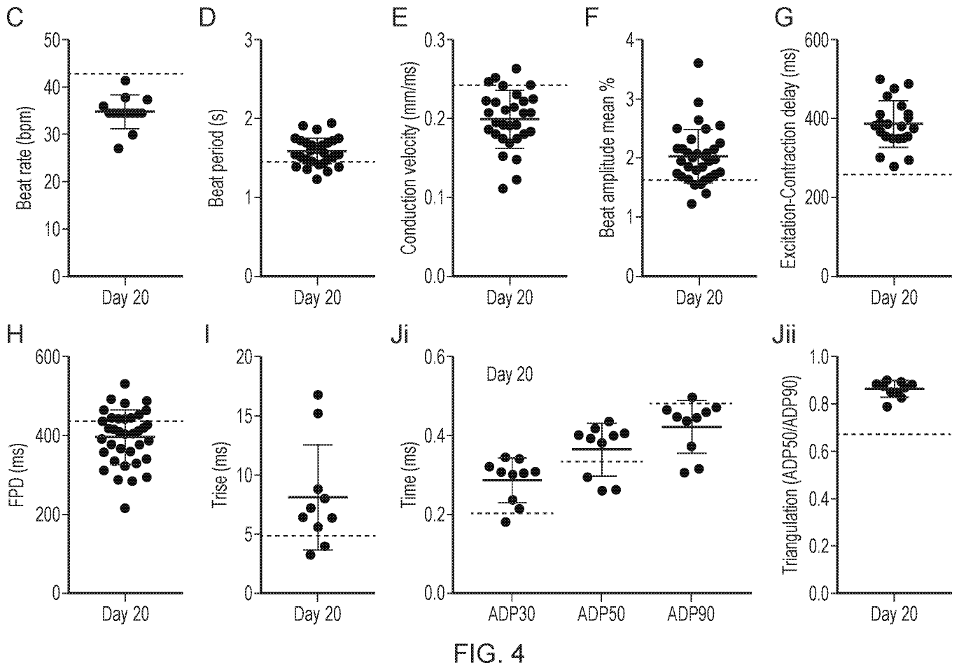

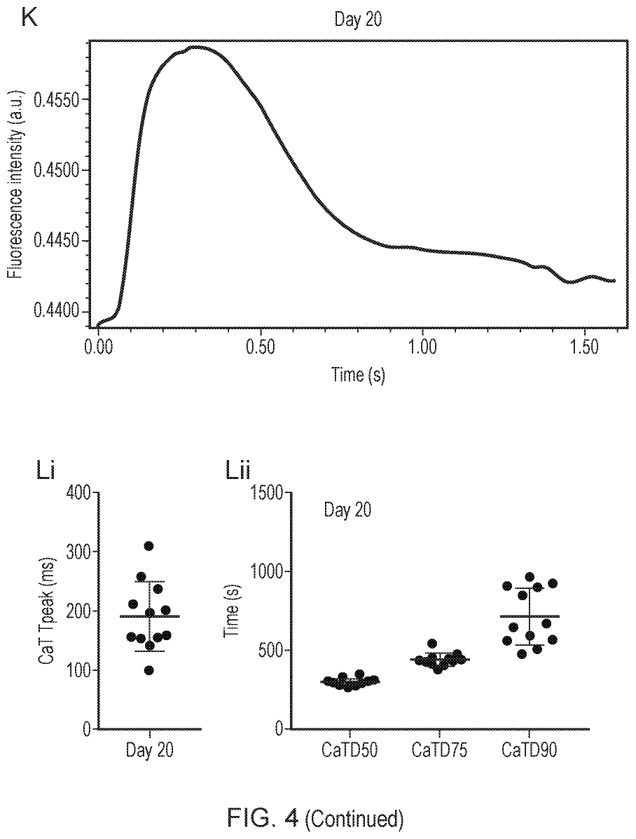

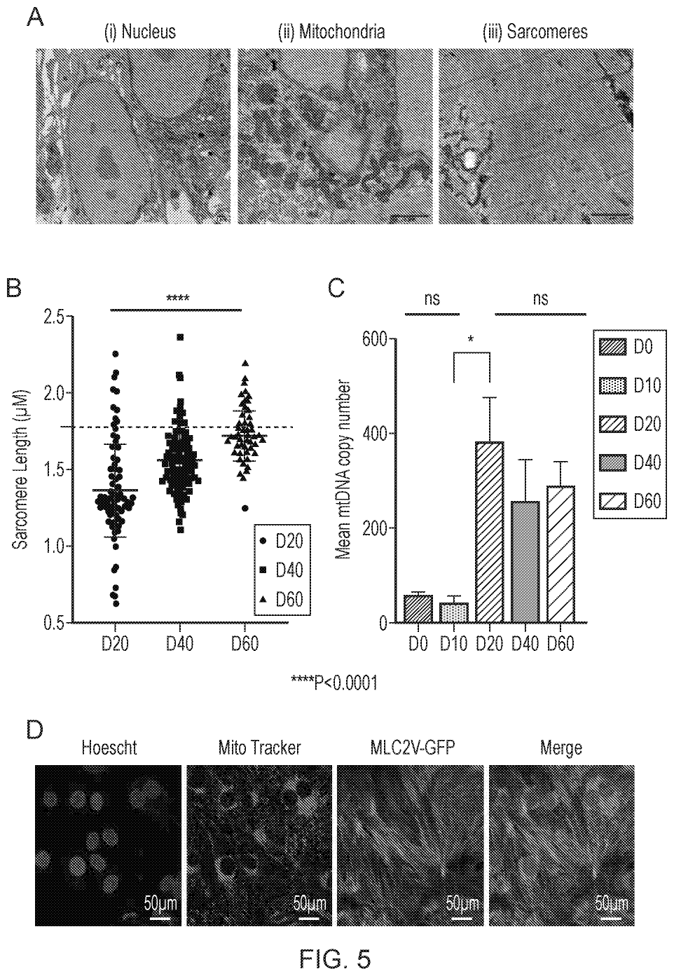

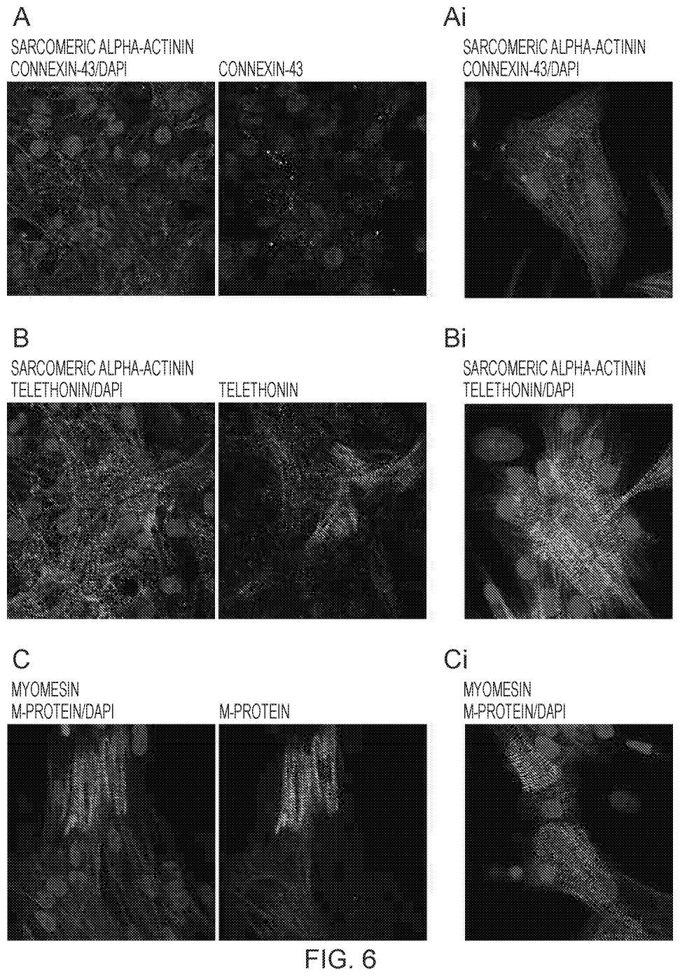

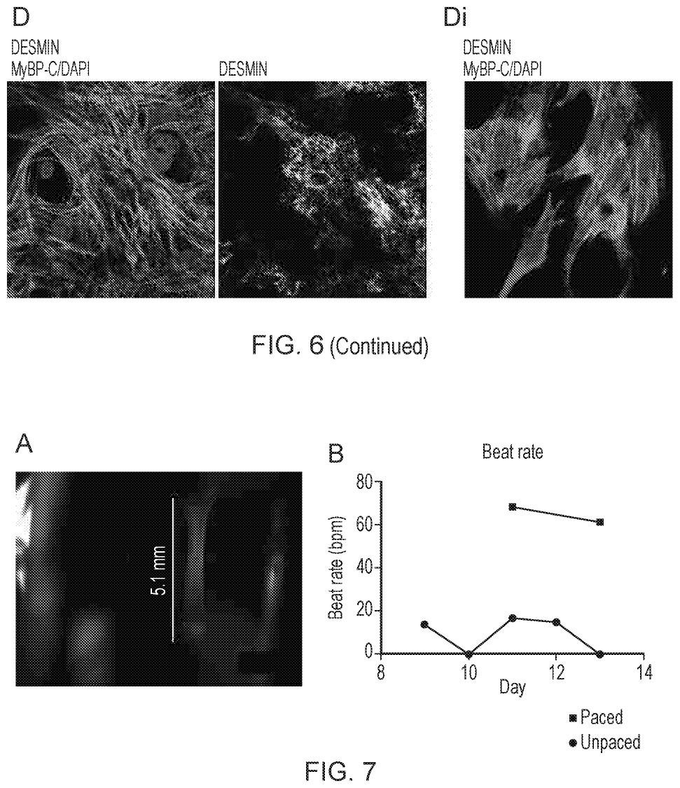

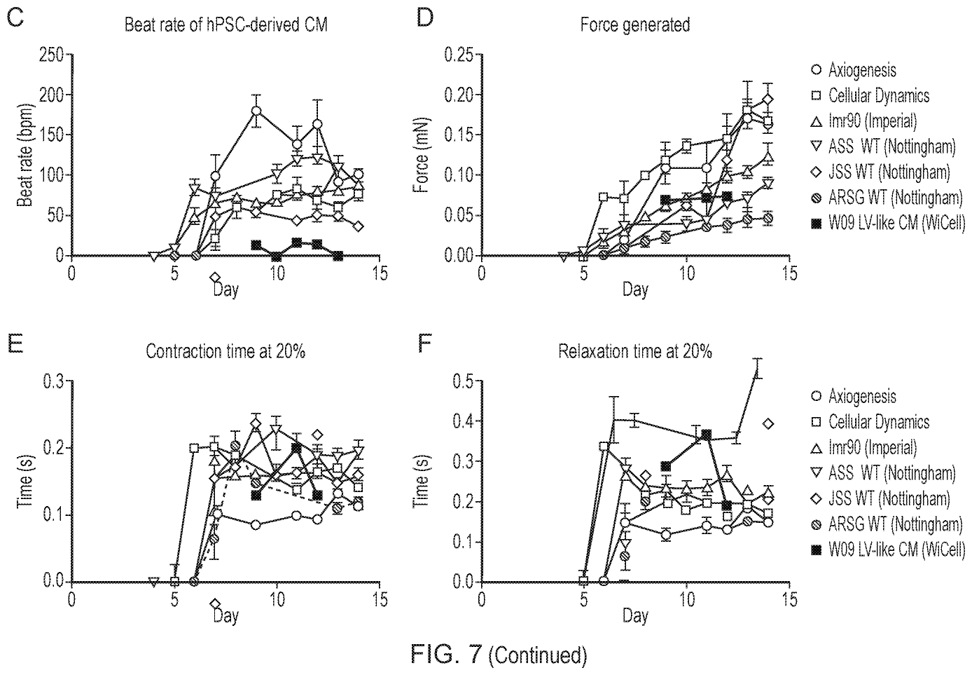

D-to-J Graphs showing the analysis of day 20 cardiomyocytes using the Axion Biosystems Maestro Pro MEA system. Average measurements for commercially available cardiomyocytes are denoted by the grey line. D Beat period analysis demonstrates the regular and uniform periodicity of beating. E Conduction velocity analysis demonstrates that LV-like cardiomyocytes have a conduction velocity close to that of neonatal cardiomyocytes (0.3 mm/ms). F Beat amplitude mean analysis demonstrates LV-like cardiomyocytes have stronger contraction force than commercially available cardiomyocytes. G Excitation-contraction delay analysis demonstrates LV-like cardiomyocytes take longer to contract post action potential initiation than commercially available cardiomyocytes likely because they exhibit a longer plateau associated with the opening/closing of slow calcium channels, typical of ventricular cardiomyocytes (see FIG. 4A). H Field potential duration (FDP) analysis demonstrates it ranged between 300 ms-500 ms. I Action potential rise time (Trise) analysis shows the fast firing ability of the LV-cardiomyocytes. Ji Action potential duration (ADP) analysis at 30% (ADP30), 50% (ADP50), and 90% (ADP90) of depolarisation shows the ADP50 and ADP90 are not very far apart in keeping with the existence of a plateau followed by a rapid repolarization, in keeping with a ventricular action potential shape. Jii Action potential triangulation as determined by the ratio between ADP50 and APD90 confirms LV-like cardiomyocytes repolarise rapidly following the plateau (in keeping with a ventricular shape) in contrast to commercially available cardiomyocytes, which have a triangular-like action potential shape, i.e. absence of a plateau. K Representative plot of the average calcium transient (CaT) of day 20 cardiomyocytes as determined using Fura-4F. Data denotes the long CaT duration. L Graphs showing analysis of the CaTs obtained from day 20 cardiomyocytes as described in F. Li CaT rise time (time to peak, Tpeak) and Lii CaT duration at 50% (CaTD50), 75% (CaTD75), and 90% (CaTD90) decay.

FIG. 5 shows the subcellular characterization of day 20 cardiomyocytes produced using the left ventricle cardiomyocyte protocol. A transmission electron microscopy (TEM) images show the ultrastructure of cardiomyocytes at day 20 of differentiation highlighting: (i) the nucleus, (ii) the mitochondria, and (iii) the sarcomeres. B Graph of the sarcomere length after 20, 40 and 60 days of differentiation denoting that at day 20 the average sarcomere length is smaller than that of a typical human adult cardiomyocyte (grey line) but that, over time, the cells increase their sarcomere length and by day 60 they have nearly the typical length of an adult ventricular cardiomyocyte. C Graph of the mean mitochondrial DNA copy number at days 0, 10, 20, 40 and 60 of differentiation denoting a sharp increase at day 20 suggesting an increased mitochondrial activity from this day onwards. D Representative confocal micrographs of cardiomyocytes at day 20 of differentiation showing mitochondria stained with MitoTracker, endogenously GFP tagged MLC2v and the live nuclear stain Hoechst. These images highlight the extensive interconnected mitochondrial network typical of neonatal cardiomyocytes.

FIG. 6 shows the cytoarchitecture and maturity characterization of day 20 cardiomyocytes produced using the left ventricle cardiomyocyte protocol. A Representative confocal micrographs showing immunostaining of the mature gap-junction marker CONNEXIN-43 and SARCOMERIC ALPHA-ACTININ, demonstrating that several cells already display mature gap junctions at day 20 of differentiation as is the case for rat neonatal cardiomyocytes (Ai). B Representative confocal micrographs showing immunostaining of the mature Z-disk marker TELETHONIN and SARCOMERIC ALPHA-ACTININ, demonstrating that at day 20 of differentiation cells exhibit similar Z-disk maturation to that seen in a rat neonatal cardiomyocytes (Bi). C Representative confocal micrographs showing immunostaining of the mature M-band marker M-PROTEIN and MYOMESIN, demonstrating that at day 20 of differentiation cells exhibit similar M-band maturation to that seen in a rat neonatal cardiomyocytes (Ci). D Representative confocal micrographs showing immunostaining of the mature cardiomyocyte associated intermediate filament DESMIN and MyBP-C, demonstrating that, as is the case for rat neonatal cardiomyocytes (Di), DESMIN can be seen concentrated around Z-disks (and connects them as is expected for mature cardiomyocytes) in addition to filamentous signals.

FIG. 7 shows the characterization of engineered heart tissues generated using day 40 cardiomyocytes produced using the left ventricle cardiomyocyte protocol. A Representative light micrograph of a EHT showing the size of the EHT generated from pole to pole. B Graph showing the beat rate of EHTs over time in culture, demonstrating that they only started beating at day 9 and thereafter their spontaneous beat rate was very low or inexistent. The EHT could however be paced (red line), demonstrating that these cardiomyocytes exhibit a relatively mature ventricular beat phenotype, since adult ventricular cardiomyocytes only beat when stimulated. C/D/E/F Graphs comparing the EHT properties of EHTs generated with LV-like cardiomyocytes (LV-EHTs, black) over time in culture in comparison to EHTs generated with other hPSC-derived cardiomyocytes (grey). C depicts the beat rate of the EHTs, confirming the unique slow rate of the LV-EHTs. D depicts the force generated confirming that LV-EHTs can generate force. E depicts the amount of time it took to reach 20% of contraction confirming that LV-EHTs exhibit an average contraction time at 20% in comparison to other EHTs tested. F depicts the amount of time it took to reach 20% of relaxation confirming it takes an average amount of time for LV-EHTs to relax in comparison to other EHTs tested.

### DETAILED DESCRIPTION OF THE INVENTION

### Left Ventricular Cardiomyocytes

As described herein, the present invention provides a population of cells comprising a significant number or proportion of left ventricular cells, particularly left ventricular cardiomyocytes.

The population of cells according to the invention has a significant level of homogeneity of left ventricular cells. Such a cell population has not been generated previously. As described in the Examples herein, the present inventors have developed a method for providing such cell populations.

By “cell population” or “population of cells” as used herein is meant more than one cell. The cells according to the invention are cardiomyocyte cells, particularly left ventricular myocytes.

Left ventricular cells may be characterised by the presence of particular markers. One such marker may be TBX5. Tbx5 is a gene that is located on the long arm of chromosome 12. Tbx5 produces a protein called T-box 5 that acts as a transcription factor. The Tbx5 gene is involved with forelimb and heart development. This gene impacts the early development of the forelimb by triggering fibroblast growth factor, FGF10. TBX5 is only expressed in the first heart field, and is therefore a LV and atrial precursor. In human adults, TBX5 expression is highest in the atrial appendages, followed by the lungs, left ventricle, and esophagus.

A further marker may be IRX4 (Iroquois-class homeodomain protein IRX-4, also known as Iroquois homeobox protein 4), which is a protein that in humans is encoded by the IRX4 gene. IRX4 is a member of the Iroquois homeobox gene family. Members of this family appear to play multiple roles during pattern formation of vertebrate embryos. Among its related pathways are heart development and cardiac progenitor differentiation. IRX4 is not sufficient for ventricular chamber formation in mice, but is required for the establishment of some components of a ventricle-specific gene expression program. In the absence of genes under the control of IRX4, ventricular function deteriorates and cardiomyopathy ensues.

A further marker may be HAND1 (Heart- and neural crest derivatives-expressed protein 1), which is a protein that in humans is encoded by the HAND1 gene.

A member of the HAND subclass of basic Helix-loop-helix (bHLH) transcription factors, the HAND1 gene is vital for the development and differentiation of three distinct embryological lineages including the cardiac muscle cells of the heart, trophoblast of the placenta, and yolk sac vasculogenesis. Most highly related to twist-like bHLH genes in amino acid identity and embryonic expression, HAND1 can form homo- and heterodimer combinations with multiple bHLH partners, mediating transcriptional activity in the nucleus.

HAND1 has a role in cardiac morphogenesis. In the third week of fetal development the rudimentary heart (bilaterally symmetrical cardiac tube) undergoes a characteristic dextral looping, forming an asymmetrical structure with bulges that represent the incipient ventricular and atrial chambers of the heart. Arising from cells derived from the primary heart field in the cardiac crescent, HAND1 goes from being expressed on both sides of the heart tube to the ventral surface of the caudal heart segment and the aortic sac, then being restricted to the outer curvature of the left ventricle in the looped heart.

In conjunction with HAND2 (a fellow bHLH transcription factor), complementary and overlapping expression patterns are thought to play a role in interpreting asymmetrical signals in the developing heart which leads to the characteristic looping. The two are implemented in cardiac development of embryos based on a crucial HAND gene dosage system. If HAND1 is over or under expressed then morphological abnormalities can form; most notable are cleft lips and palates. Expression was modelled with a knock-in of phosphorylation to turn on and off gene expression which induced the craniofacial abnormalities. HAND1 has been associated with hypoplastic left heart syndrome.

A further marker may be ventricular myosin light chain-2 (MLC-2v), which refers to the ventricular cardiac muscle form of myosin light chain 2 (MYL2). MLC2v is strongly expressed in the ventricular myocardium. MLC-2v plays an essential role in early embryonic cardiac development and function and represents one of the earliest markers of ventricular specification. During early development (E7.5-8.0), MLC-2v is expressed within the cardiac crescent. The expression pattern of MLC-2v becomes restricted to the ventricular segment of the linear heart tube at E8.0 and remains restricted within the ventricle into adulthood.

In one aspect of the invention the population of cells comprises at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% cells that are double positive for the markers HAND1 and MLC2v. In one aspect the population of cells comprises at least about 85, 90, 95, 96, 97, 98, 99 or 100% cells that are double positive for the markers HAND1 and MLC2v.

In one aspect of the invention the population of cells comprises at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% cells that are positive for the markers TBX5, and HAND1 and MLC2v. In one aspect the population of cells comprises at least about 85, 90, 95, 96, 97, 98, 99 or 100% cells that are positive for the markers TBX5, and HAND1 and MLC2v.

In one aspect of the invention the population of cells comprises at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% cells that are positive for the markers TBX5, HAND1, MLC2v and IRX4. In one aspect the population of cells comprises at least about 85, 90, 95, 96, 97, 98, 99 or 100% cells that are positive for the markers TBX5, HAND1, MLC2v and IRX4.

In one aspect of the invention the population of cells comprises at least about 65, 70, 75, 80, 85, 90, 95 or 100% cells that are double positive for the markers HAND1 and MLC2v.

In one aspect of the invention the population of cells comprises at least about 65, 70, 75, 80, 85, 90, 95 or 100% cells that are positive for the markers TBX5, and HAND1 and MLC2v.

In one aspect of the invention the population of cells comprises at least about 65, 70, 75, 80, 85, 90, 95 or 100% cells that are positive for the markers TBX5, HAND1, MLC2v and IRX4.

In one aspect the population of cells which comprises at least about 85% cells that are double positive for the markers HAND1 and MLC2v.

In one aspect the population of cells which comprises at least about 90% cells that are double positive for the markers HAND1 and MLC2v.

In one aspect the population of cells which comprises at least about 95% cells that are double positive for the markers HAND1 and MLC2v.

In one aspect the population of cells which comprises at least about 85% cells that are positive for the markers TBX5, and HAND1 and MLC2v.

In one aspect the population of cells which comprises at least about 90% cells that are positive for the markers TBX5, and HAND1 and MLC2v.

In one aspect the population of cells which comprises at least about 95% cells that are positive for the markers TBX5, and HAND1 and MLC2v.

In one aspect the population of cells which comprises at least about 85% cells that are positive for the markers TBX5, HAND1, MLC2v and IRX4.

In one aspect the population of cells which comprises at least about 90% cells that are positive for the markers TBX5, HAND1, MLC2v and IRX4.

In one aspect the population of cells which comprises at least about 95% cells that are positive for the markers TBX5, HAND1, MLC2v and IRX4.

In one aspect of the invention the cells may be positive for HAND1 and/or MLC2v.

Methods for testing for the presence of such markers will be routine for those of skill in the art. For example, FACS analysis or immunofluorescence could be used as described in the present Examples.

Left ventricular cells are distinct from right ventricular cells. The left and right ventricular cells are derived from separate and distinct cell lineages.

In one aspect of the invention as described herein, the population of cells may be further purified in order to increase the percentage of left ventricle cells in said population. The left ventricle cells may be selected from the population using methods known in the art, for example using cell surface markers or other methods, such as selecting from genome edited cell lines whereby a subset of cells based on reporter tags is purified, or killing the unwanted cells based on killer-gene selection.

In one aspect, purification may be performed for example via metabolic selection. Metabolic selection is a method which relies on removing glucose from the media and replacing it with lactic acid; very few cell types, including cardiomyocytes, can survive under these conditions. Metabolic selection may be used for a long period to enrich for left ventricular cardiomyocytes. In one example, cells may be cultured in a metabolic selection medium between day 10 and day 12 of the culture protocol as described herein.

In a further aspect, purification may be performed by flow cytometry or via the use of beads on the basis of a surface marker gene which detects the said population according to the invention. Purification may also require the use of a reporter cell line where, for example, the HAND1 and MLC2V loci are tagged with a florescent gene sequence and cells are purified based on being double positive for the reporter genes tagged to the HAND1 and MLC2V loci.

In one aspect of the invention as described herein, the population of cells may be further matured by adding supplements to the culture media which may include, but are not limited to, triiodothyronine (T3) (16028, Cayman), insulin-like growth factor 1 (IGF-1) (11271, Sigma-Aldrich), dexamethasone (Dex) (D4902, Sigma-Aldrich), fatty acids such as palmitic acid (810105P, Sigma Aldrich), oleic acid (O3008, Sigma Aldrich), and linoleic acid (L9530, Sigma Aldrich).

In one aspect, maturation may also be promoted by growing cells on alternative substrates or combinations of such, e.g. fibronectin (PHE0023, Thermo Fisher), laminin 511 (LN511, BioLamina), laminin 521 (LN521, BioLamina).

In one aspect of the invention as described herein, a great proportion of the population, for example at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% of the population, exhibits various hallmarks of maturity, for example cells display a well-developed myofibrillar arrangement including defined Z-disks, a conduction velocity close to that of neonatal cardiomyocytes, a sarcomere length approaching that of fully mature ventricle cardiomyocytes, extensive interconnected mitochondrial networks typical of neonatal cardiomyocytes, and express various maturity sarcomere markers such as SARCOMERIC ALPHA-ACTININ, M-PROTEIN and TELETHONIN.

### Disorders of the Left Ventricle

The invention as described herein may be used in the treatment or prevention of a disorder of the left ventricle.

The left ventricle is one of four chambers of the heart. It is located in the bottom left portion of the heart below the left atrium, separated by the mitral valve. As the heart contracts, blood eventually flows back into the left atrium, and then through the mitral valve, whereupon it next enters the left ventricle. From there, blood is pumped out through the aortic valve into the aortic arch and onward to the rest of the body. The left ventricle is the thickest of the heart's chambers and is responsible for pumping oxygenated blood to tissues all over the body. By contrast, the right ventricle solely pumps blood to the lungs.

Various conditions may affect the left ventricle and interfere with its proper functioning. The most common is left ventricular hypertrophy, which causes enlargement and hardening of the muscle tissue that makes up the wall of the left ventricle, usually as a result of uncontrolled high blood pressure. Another condition that may impact this area is left ventricular non-compaction cardiomyopathy, in which the muscle tissue surrounding the left ventricle is spongy or “non-compacted”.

In one aspect the cells according to the present invention may be used to treat or prevent damage to the left ventricle. Specifically, the present invention could help identify cardiotoxic drugs or be used as a therapy to treat hearts damaged by these drugs. For example, a left ventricular disorder may involve or result from drug-induced damage to the heart, for example drug-induced heart failure. Some drugs or therapies may lead to damage to the heart, for example the left ventricle, as a side effect to their intended therapeutic use. This may apply, for example, to some cancer therapies or drugs.

In one aspect of the present invention the disorder of the left ventricle may be selected from myocardial infarction, heart failure, left ventricular hypertrophy, hypoplastic left heart syndrome and left ventricular non-compaction cardiomyopathy (LVNC).

In one aspect of the present invention the disorder of the left ventricle is myocardial infarction.

Myocardial infarction (MI), commonly known as a heart attack, occurs when a portion of the heart is deprived of oxygen due to blockage of a coronary artery. Coronary arteries supply the heart muscle (myocardium) with oxygenated blood. Without oxygen, muscle cells served by the blocked artery begin to die (infarct). The cause of myocardial infarction is often atherosclerosis, a buildup of fatty plaque and other material inside the artery. The plaque is covered by a lining of fibrous material. That lining can rupture, allowing the plaque to be released and a blood clot to form. Myocardial infarction is virtually synonymous with left ventricular infarction, as almost all myocardial infarctions affect the left ventricle. The heart has 3 coronary arteries, 2 of them always feed the left ventricle, part of the right coronary artery can however feed exclusively the right ventricle. Only 25% of heart attacks affect the right coronary artery and of these 1/12 affects the right ventricle only.

In one aspect of the present invention the disorder of the left ventricle is left ventricular hypertrophy.

Left ventricular hypertrophy is enlargement and thickening (hypertrophy) of the walls of the left ventricle. Left ventricular hypertrophy can develop in response to, for example, high blood pressure or a heart condition that causes the left ventricle to work harder.

In one aspect of the present invention the disorder of the left ventricle is heart failure.

Heart failure, sometimes known as congestive heart failure, occurs when the heart muscle doesn't pump blood as well as it should. Certain conditions, such as narrowed arteries in the heart (coronary artery disease) or high blood pressure, gradually leave the heart too weak or stiff to fill and pump efficiently. Heart failure often develops after other conditions have damaged or weakened the heart. However, the heart doesn't need to be weakened to cause heart failure. It can also occur if the heart becomes too stiff.

In heart failure, the main pumping chambers of your heart (the ventricles) may become stiff and not fill properly between beats. In some cases of heart failure, the heart muscle may become damaged and weakened, and the ventricles stretch (dilate) to the point that the heart can't pump blood efficiently throughout your body.

Over time, the heart can no longer keep up with the normal demands placed on it to pump blood to the rest of your body.

An ejection fraction is an important measurement of how well the heart is pumping and is used to help classify heart failure and guide treatment. In a healthy heart, the ejection fraction is 50 percent or higher, meaning that more than half of the blood that fills the ventricle is pumped out with each beat. Heart failure can occur even with a normal ejection fraction. This happens if the heart muscle becomes stiff from conditions such as high blood pressure. Heart failure can involve the left side (left ventricle), right side (right ventricle) or both sides of your heart. Generally, heart failure begins with the left side, specifically the left ventricle.

In one aspect of the present invention the disorder of the left ventricle is left ventricular hypertrophy.

In left ventricular non-compaction cardiomyopathy (LVNC) the lower left chamber of the heart, called the left ventricle, contains bundles or pieces of muscle that extend into the chamber. These pieces of muscles are called trabeculations. During development, the heart muscle is a sponge-like network of muscle fibers.

As normal development progresses, the trabeculations become compacted transforming the heart muscle from sponge-like to smooth and solid. LVNC occurs when compaction does not occur. These trabeculations typically occur at the bottom of the heart called the apex but can be seen anywhere in the left ventricle. Individuals with LVNC may also have another type of heart muscle disease (hypertrophic cardiomyopathy, dilated cardiomyopathy or restrictive cardiomyopathy).

In one aspect of the present invention the disorder of the left ventricle is hypoplastic left heart syndrome.

Hypoplastic left heart syndrome (HLHS) is a birth defect that affects normal blood flow through the heart. As the baby develops during pregnancy, the left side of the heart does not form correctly. Hypoplastic left heart syndrome is one type of congenital heart defect. In HLHS the left ventricle of the heart does not develop properly so is much smaller than usual. The mitral valve between the left ventricle and the upper left filling chamber (left atrium) is often closed or very small.

The cell population according to the present invention may be used as a cell therapy for the treatment or prevention of a disorder of the left ventricle as described herein. Cell therapy is therapy in which cellular material is injected, grafted or implanted into a patient; this generally means intact, living cells.

In the present case, the population of cells according to the present invention may be administered to a subject who has a disorder of the left ventricle. The invention includes use of the cell population according to the invention as a cell therapy.

In one aspect the invention provides a cell population according to the invention for use as a cell therapy for treating or preventing a disorder of the left ventricle.

The invention also provides use of the cell population according to the invention as a cell therapy for treating or preventing a disorder of the left ventricle.

### Method for Producing a Cell Population

In one aspect of the invention is provided a method for producing a population of cells as described herein.

The invention provides an improved method for generating a population of cells comprising an increased number or proportion of left ventricular cardiomyocytes in particular, i.e. at a level that may be useful practically and therapeutically. The advantages of the present invention over protocols known in the art are rapid achievement of good quality cell populations, and attainment of over 85% of LV cardiomyocytes. The cells produced according to the present invention are more mature than cells produced using methods known in the art under equivalent monolayer conditions.

The method according to the invention comprises the step of culturing a pluripotent stem cell in a medium comprising a retinoic acid receptor antagonist or inverse agonist. It was not obvious from previous work in the art that antagonising the retinoic acid receptor would lead to an increase in left ventricular cardiomyocyte differentiation. This was a surprising finding by the present inventor.

As such, the invention provides a method for producing a population of cells according to the invention, wherein said method comprises the step of culturing pluripotent stem cells in a medium comprising a retinoic acid receptor antagonist or inverse agonist.

By “pluripotent stem cell” is meant a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system).

In a preferred aspect the pluripotent stem cell is an embryonic stem cell, most preferably a human embryonic stem cell.

As is known in the art, embryonic stem cells are pluripotent stem cells derived from early embryos. Embryonic stem cell lines (ES cell lines) are cultures of cells derived from the epiblast cells of the inner cell mass (ICM) of a blastocyst or earlier morula stage embryos. A blastocyst is an early stage embryo that is approximately five to 7 days old in humans and is composed of 100-300 cells. ES cells are pluripotent and give rise during development to all derivatives of the three primary germ layers: ectoderm, endoderm and mesoderm. In other words, they can develop into each of cell types of the adult body.

In an alternative embodiment the pluripotent stem cell is an induced pluripotent stem cell, most preferably a human induced pluripotent stem cell.

Induced pluripotent stem cells are a type of pluripotent stem cell artificially prepared from a non-pluripotent cell, typically an adult somatic cell, or terminally differentiated cell, such as a fibroblast, a hematopoietic cell, a myocyte, a neuron, an epidermal cell, or the like, by inserting certain genes or non-integrating mRNAs or chemicals, referred to as reprogramming factors.

Cells may be transduced, transfected, electroporated or nucleofected with any one or a combination of the transcription factors SOX2 (SRY-related HMG-box 2), OCT4 (Octamer-binding transcription factor 4), KLF4 (Kruppel-Like Factor 4), and c-MYC (V-myc avian myelocytomatosis viral oncogene homolog), L-MYC, N-MYC, NANOG, LIN28, SALL4, UTF1, TBX3, inhibitors of p53 and/or p21 and/or the presence of epigenetic modifying drugs such as 5′-azacytidine and RG108. One skilled in the art will appreciate that this list is not exhaustive, and is merely an example of some of the factors or combination of factors that have been used to generate induced pluripotent stem (iPS) cells resembling hES cells. These factors affect conversion of non-pluripotent cells into iPS cells. It is known in the art that adult mice can be derived from iPS cells. These reprogrammed cells acquire ES cell-like properties, and therefore have the potential to generate any tissue (Boland, et al. (2009) Nature 461:91-94; Quinlan, et al. (2011) Cell Stem Cell 9:366-373).

One skilled in the art would be aware of methods for culturing, isolating or producing embryonic stem cells.

For example, hES cells can be obtained from blastocysts, for example using methods as set out in Thomson, et al. (1995) Proc. Natl. Acad. Sci. USA 92:7844-7848; Thomson, et al. (1998) Science 282:1145; Thomson & Marshall (1998) Curr. Top. Dev. Biol. 38:133-165; Reubinoff, et al. (2000) Nat. Biotechnol. 18:399-404; Chen and Egli et al., Cell Stem Cell, 2009 Feb. 6; 4.

Established ES cell lines are also available. Various hES cell lines are known and conditions for their growth and propagation have been defined, for example, hES cell lines Shef6, WA01, WA07, WA09, WA13 and WA14. Any ES cells or ES cell lines are suitable for use according to the present invention.

ES cells may be derived from a blastocyst, by culturing the inner cell mass of a blastocyst, or obtained from cultures of established cell lines. Thus, as used herein, the term “ES cells” can refer to inner cell mass cells of a blastocyst, ES cells obtained from cultures of cells from the inner cell mass, and ES cells obtained from cultures of ES cell lines.

iPS cells may be obtained by various methods. For example, see the method of Takahashi, et al. (2007) Cell 126(4):663-76). The iPS cells are morphologically similar to hES cells, and express various hES cell markers.

Human embryonic stem cells may be defined by the presence of several transcription factors and cell surface proteins as determined by immunohistochemistry and/or flow cytometry. Suitable transcription factor markers include OCT4, NANOG, and SOX2, and suitable antigen markers include the glycolipids SSEA-1 (absence thereof), SSEA3 and SSEA4 and the keratan sulphate antigens TRA-1-60 and TRA-1-81. Such methods are routine in the art.

iPS cells may be defined by the presence of several transcription factors and cell surface proteins as determined by immunohistochemistry and/or flow cytometry. Suitable transcription factor markers include OCT4, NANOG, and SOX2, and suitable antigen markers include the glycolipids SSEA-1 (absence thereof), SSEA3 and SSEA4 and the keratan sulphate antigens TRA-1-60 and TRA-1-81. Such methods are routine in the art.

Pluripotency of embryonic stem cells can be confirmed by spontaneous or directed differentiation in vitro or by injecting approximately 0.5-10×10⁶ cells into the rear leg muscles of 8-12 week old male SCID mice, generating teratomas that demonstrate at least one cell type of each of the three germ layers.

Suitable cells will be known to those of skill in the art. For example, WA09 cells, WA01 cells, AICS cells (iPSCs) or even disease cell lines such as Progeria cells (iPSCs) may be used.

Suitable retinoic acid receptor antagonists or inverse agonists are known in the art.

An antagonist is a type of receptor ligand or drug that blocks or dampens a biological response by binding to and blocking a receptor rather than activating it like an agonist. They are sometimes called blockers; examples include alpha blockers, beta blockers, and calcium channel blockers. In pharmacology, antagonists have affinity but no efficacy for their cognate receptors, and binding will disrupt the interaction and inhibit the function of an agonist or inverse agonist at receptors. Antagonists mediate their effects by binding to the active site or to the allosteric site on a receptor, or they may interact at unique binding sites not normally involved in the biological regulation of the receptor's activity. Antagonist activity may be reversible or irreversible depending on the longevity of the antagonist—receptor complex, which, in turn, depends on the nature of antagonist—receptor binding. The majority of drug antagonists achieve their potency by competing with endogenous ligands or substrates at structurally defined binding sites on receptors.

An antagonist is distinct from an inverse agonist. An inverse agonist is an agent that binds to the same receptor as an agonist but induces a pharmacological response opposite to that agonist. A neutral antagonist has no activity in the absence of an agonist or inverse agonist but can block the activity of either. Inverse agonists have opposite actions to those of agonists but the effects of both of these can be blocked by antagonists. An agonist increases the activity of a receptor above its basal level, whereas an inverse agonist decreases the activity below the basal level.

In one aspect of the invention AGN193109 (sc-210768, Santa Cruz Biotechnology) may be used, which is sold as a high affinity pan-retinoic acid receptor (RAR) antagonist.

In one aspect of the invention BMS493 (available from e.g. StemCell Technologies, Tocris, Merck, R&D Systems) may be used. BMS493 is a pan-RA inverse agonist.

Base media that may be used according to the invention as described herein include, but are not limited to, StemPro-34, Dulbecco's Modified Eagle's Medium (DMEM), Ham's F10 medium, Ham's F12 medium, Advanced DMEM, Advanced DMEM/F12, minimal essential medium, DMEM/F-12, DMEM/F-15, Liebovitz L-15, RPMI 1640, Iscove's modified Dubelcco's media (IMDM), OPTI-MEM SFM (Invitrogen Inc.), N2B27, MEF-CM and defined basal ESC medium, ExVivo 10, ESGrow or a combination thereof.

In one aspect the medium is RPMI1640 medium, for example available from LifeTech.

In one aspect the medium comprises a B27 supplement without insulin, for example available for example from Gibco (ThermoFisher Scientific MA, USA).

In one aspect the medium may comprise a Wnt. Wnt signaling pathways are a group of signal transduction pathways which begin with proteins that pass signals into a cell through cell surface receptors. Wnt signaling pathways use either nearby cell-cell communication (paracrine) or same-cell communication (autocrine).

In one aspect the medium may comprise a Wnt agonist.

In one aspect the medium may comprise a glycogen synthase kinase-3 (Gsk3) inhibitor. For example, the small molecule Chiron, CHIR99021 (a Gsk3 inhibitor), may be present in the medium. Suitable sources of Chiron for use according to the invention are commercially available, for example from Selleck Chem (S2924).

In one aspect the medium may contain about 1-12 μM/ml Chiron, for example about 2-5 or 2-3 μM/ml Chiron.

In one aspect the medium may also comprise a BMP. Originally discovered by their ability to induce the formation of bone and cartilage, BMPs are now considered to be important in orchestrating tissue architecture throughout the body. Seven BMPs were discovered originally. Of these, six (BMP2 to BMP7) belong to the Transforming growth factor beta superfamily of proteins. BMP1 is a metalloprotease. Thirteen further BMPs have since been discovered, bringing the total to twenty.

In one aspect the medium comprises BMP4. Suitable sources of BMP4 for use according to the invention are commercially available, for example from R&D Systems (314-BP-010).

In one aspect the medium may contain about 1-10 ng/ml BMP, e.g. BMP4, for example about 1 to about 6 ng/ml, or about 3 to about 5 ng/ml BMP, e.g. BMP4.

In one aspect the medium may comprise an Activin, Nodal or TGFβ. For example, exogenous Activin such as Activin A, Activin AB and/or Activin B may be present in the cell medium. Suitable sources of Activin for use according to the invention are commercially available, for example from R&D Systems (cat no. 338-AC/CF) or Peprotech (cat no. 120-14).

In one aspect the medium comprises Activin A.

In one aspect the medium may contain about 1-10 ng/ml Activin, e.g. Activin A, for example about 3 to about 10 ng/ml Activin. In one aspect the medium may contain about 5 ng/ml Activin, e.g. Activin A.

Nodal is a secretory protein that in humans is encoded by the NODAL gene which is located on chromosome 10. It belongs to the transforming growth factor beta (TGF-β) superfamily. In one aspect the medium may comprise Nodal. For example, exogenous Nodal may be present in the cell medium. Suitable sources of Nodal for use according to the invention are commercially available, for example from R&D Systems (cat no. 3218-ND/CF). One skilled in the art may be able to determine suitable amounts of Nodal that may be included in the medium.

Transforming growth factor beta (TGF-β) is a multifunctional cytokine belonging to the transforming growth factor superfamily. In one aspect the medium may comprise TGF-β. For example, exogenous TGF-β such as TGF-β 1, TGF-β 2 and/or TGF-β 3, may be present in the cell medium. Suitable sources of TGF-β for use according to the invention are commercially available, for example from R&D Systems (cat no. 7754-BH/CF). One skilled in the art may be able to determine suitable amounts of TGF-β that may be included in the medium.

In one aspect the medium may also comprise FGF. The FGFs are a family of growth factors with members involved in angiogenesis, wound healing, embryonic development and various endocrine signalling pathways. The term “FGF” as used herein is intended to encompass any member of the FGF family, for example FGF1, 2, 3, 4, 5, 6, 7, 8, 9, 10, 11, 12, 13, 14, 15, 16, 17, 18, 19, 20, 21, 22 or 23. FGFs are commercially available.

In one aspect the FGF is FGF2. Suitable sources of FGF2 for use according to the invention are commercially available, for example from R&D Systems (233-FB-025).

In one aspect the medium may contain about 1-20 ng/ml FGF, e.g. FGF2, for example about 3 to about 10 ng/ml. In one aspect the medium may contain about 5 ng/ml FGF, e.g. FGF2.

In one aspect the medium may also comprise a Wnt inhibitor. Such inhibitors will be known to one skilled in the art. In one aspect such inhibitor may be, for example, IWR1, IWP2, C59 or DKK.

In one aspect the medium may also comprise L-ascorbic acid (also referred to herein as L-AA).

In one aspect the medium does comprise vitamin A. In one aspect the medium does not comprise vitamin A.

One skilled in the art may be able to determine suitable amounts of such factors to be added to the medium.

The invention encompasses a cell population obtained or obtainable by the methods as described herein. Such a cell population may be used in any of the methods or uses as described herein. In one aspect the invention encompasses producing a population of cells as described herein according to a method of the invention, and then using said cells for treating or preventing a disorder of the left ventricle as described herein.

In one aspect the method comprises culturing the pluripotent cells in a medium as described herein from day 0 of differentiation of the pluripotent stem cells. Day 0 may be taken as the first day in which differentiation of the pluripotent stem cell is initiated.

As described herein, cells may be plated at a given density and then left to reach a certain confluency while being grown in pluripotency maintenance medium. One skilled in the art may be able to determine suitable amounts of cells and the time needed to achieve an appropriate density and compaction.

A suitable method for providing the cell population according to the present invention is as described in the present Examples.

In one aspect the invention provides a method for culturing left ventricular cardiomyocytes, wherein said method comprises culturing embryonic stem cells with a Wnt/Wnt agonist/GSK3b inhibitor (preferably Chiron), BMP (preferably BMP4), Activin (preferably Activin A) and FGF (preferably FGF2). In one aspect said culturing may be for a period of about 1 or 2 days, preferably about 1 day. The amounts of each component may be as described above.

In one aspect the cells may be cultured with B27 (preferably without insulin) and a retinoic acid inhibitor (preferably AGN193109) or inverse agonist. Such a medium is referred to herein as “heart medium 1”. Optionally L-ascorbic acid may be added. The Wnt inhibitor IWR is added on day 2.

In one aspect the cells are incubated in heart medium 1 for about 8 days. The first day of incubation in heart medium 1 is referred to as day 0. The cells may be pluripotent on day 0. The cells are incubated with the factors described above (Wnt/Wnt agonist/GSK3b inhibitor (preferably Chiron), BMP (preferably BMP4), Activin (preferably Activin A) and FGF (preferably FGF2) on days 0 and 1, and optionally 2.

In one aspect the medium is changed on about day 8 to a medium comprising B27 which does not comprise vitamin A, but which may comprise insulin. Optionally, the medium may comprise retinoic acid inhibitor (preferably AGN193109) or inverse agonist. Such a medium is referred to herein as “heart medium 2”. Optionally, heart medium 2 may comprise L-ascorbic acid.

In one aspect the cells are incubated in heart medium 2 for about 2 days, i.e. until day 10-12.

In one aspect the medium is changed between day 10-12 to a medium devoid of glucose and comprising B27 (which does not comprise vitamin A, but which may comprise insulin), L-lactic acid, plus or minus a retinoic acid inhibitor (preferably AGN193109) or inverse agonist, and optionally L-ascorbic acid. Such a medium is referred to herein as “heart medium 3”. In one aspect the cells may be cultured in heart medium 3 for a period of 2-4 days, e.g. between day 10 to day 12.

In one aspect the medium is changed on about day 12-14 to “heart medium 2”. Optionally L-ascorbic acid may be added. Optionally, a retinoic acid inhibitor (preferably AGN193109) or inverse agonist may be added. In one aspect the cells are incubated in heart medium 2 thereafter until experiment is terminated.

In one aspect the basal medium may be RPMI medium.

In one aspect the media may be as follows:

Heart medium 1:
1. RPMI 1640 medium containing glucose and L-glutamine.
2. B27 supplement minus insulin (preferably 1 ml per 50 ml RPMI).
3. AGN193109 (preferably at a final concentration of 20-200 nM).

Heart medium 2:
1. RPMI 1640 medium containing glucose and L-glutamine.
2. B27 supplement minus vitamin A (preferably 1 ml per 50 ml RPMI).
3. Optionally AGN193109 (preferably at a final concentration of 20-200 nM).
   Heart medium 3:
1. RPMI 1640 medium containing L-glutamine but NO glucose.
2. B27 supplement minus vitamin A (preferably 1 ml per 50 ml RPMI).
3. Optionally AGN (preferably at a final concentration of 20-200 nM).
4. L-lactic acid (preferably 4 mM)

The method may comprise about 20 or more days of culturing following day 0.

In one aspect of the invention the protocol in Table 1 below may be followed:

TABLE 1

| Day | Heart medium | Additional factors |
|---|---|---|
| 0 | 1 | Activin A, FGF2, BMP4, Chiron |
| 1 | 1 | — |
| 2 | 1 | L-ascorbic acid and IWR |
| 3 | — | — |
| 4 | 1 | L-ascorbic acid |
| 5 | — | — |
| 6 | 1 | L-ascorbic acid |
| 7 | — | — |
| 8 | 2 | L-ascorbic acid, AGN193109 |
| 9 | — | — |
| 10 | 3 | L-ascorbic acid, AGN193109 |
| 11 | — | — |
| 12 | 2 | L-ascorbic acid, AGN193109 |
| 13 | — | — |
| 14 | 2 | L-ascorbic acid, AGN193109 |
| 15 | Split cells into Stem cell | Rock inhibitor |
| | technologies support media | |
| 16 | 2 | L-ascorbic acid, AGN193109 |
| 17 | — | — |
| 18 | 2 | L-ascorbic acid, AGN193109 |
| 19 | — | — |
| 20 | 2 | L-ascorbic acid, AGN193109 |

In one aspect the cells may be cultured in heart medium 2 with L-ascorbic acid for a period of 5, 10, 15, 20, 25, 30, 35, 40, 45, 50, 55 or 60 days following day 20 of the protocol. Heart medium 2 plus L-ascorbic acid may be given every other day.

### Functional Characterisitics of Cells

As described herein, the cells generated according to the invention may have functional characteristics or properties associated with ventricular cardiomyocytes, particularly left ventricular cardiomyocytes.

As supported by the present Examples, in one aspect the cells may demonstrate one or more of the following characteristics:

1. Ventricular action potential shape.
2. Slow beat rates and/or beat periodicity seen already at day 20 and becoming more pronounced with time in culture, especially when cells are used to generate EHTs. The slower the beat rate the more mature the cells, i.e. a slower beat rate, or absence of spontaneous beating is desirable. Mature ventricular cardiomyocytes do not beat spontaneously, they beat only when stimulated. The present invention facilitates production of cells with a slower, and hence improved, beat rate, but importantly the cells can beat when stimulated.
3. Conduction velocity characteristic of neonatal ventricular cells. A conduction velocity close to that of adult cardiomyocytes is desirable, but having the conduction velocity of neonatal cardiomyocytes (0.3 mm/ms) is already very good. The present invention provides cells with a conduction velocity of about 0.3 mm/ms, which is an improvement over previous methods disclosed in the art, which have resulted in conduction velocities of about 0.04 mm/ms. The invention therefore provides a method for improving the conduction velocity of the cell population.
4. Beat amplitude mean higher than that of commercially available cardiomyocytes. The invention therefore provides a method for improving the force of the cell population.
5. Excitation-contraction delay is longer than that of commercially available cardiomyocytes likely because LV-like cardiomyocytes exhibit a longer plateau associated with the opening/closing of slow calcium channels. This plateau is typical of ventricular cardiomyocytes.
6. Field potential duration is long (about 400 ms) in keeping with the long action potential duration of ventricular cells.
7. Action potential rise time (Trise) is fast and the action potential duration and/or action potential triangulation is in keeping to what is expected of ventricular cells.
8. Calcium transients (CaTs) are close to those seen in human adult ventricular cardiomyocytes.
9. CaT rise time (time to peak, Tpeak) and CaT duration is slower than that of mature ventricle cells but an improvement over previous methods disclosed in the art.

In one aspect the cells according to the invention may be paced. This feature has particular utility in the context of engineered heart tissue, as described herein. The present Examples show that the cardiomyocytes can be used to generate EHTs which exhibit a relatively mature ventricular beat phenotype (i.e. low or absence of beating), since adult ventricular cardiomyocytes only beat when stimulated. In one aspect the amount of force generation of the cells while spontaneously beating according to the invention, for example in the context of engineered heart tissue, is in line with that of other cardiomyocytes generated commercially or according to methods previously disclosed in the art. Similarly, the contraction and relaxation time of the cells according to the invention while spontaneously beating, for example in the context of engineered heart tissue, is in line with that of other cardiomyocytes generated commercially or according to methods previously disclosed in the art.

Methods according to the present invention may provide a population of cells with any one or more of these characteristics.

The invention also provides a method for improving the maturity of the cell population in a short period of time (20 days). Markers of maturity may be improved, for example with respect to sarcomere organisation, length and function. The present invention facilitates the production of cells with sarcomere length characteristic of adult cardiomyocytes.

The cells according to the invention may also display the mature gap-junction marker CONNEXIN-43, Sarcomeric ALPHA-ACTININ, the mature z-disk marker

TELETHONIN, the mature M-band marker M-PROTEIN, and/or the mature cardiomyocyte associated intermediate filament DESMIN.

The cells according to the invention may also display an extensive interconnected mitochondrial network typical of neonatal cardiomyocytes. The present invention also facilitates the rapid activation of mitochondrial function, as per the increased activation of mitochondrial DNA seen at day 20, a feature of an active metabolism.

### Subject

In a preferred aspect of the present invention, the subject is a mammal, preferably a cat, dog, horse, donkey, sheep, pig, goat, cow, mouse, rat, rabbit or guinea pig, but most preferably the subject is a human.

As defined herein “treatment” refers to reducing, alleviating or eliminating one or more symptoms of the disease which is being treated, relative to the symptoms prior to treatment.

“Prevention” (or prophylaxis) refers to delaying or preventing the onset of the symptoms of the disease. Prevention may be absolute (such that no disease occurs) or may be effective only in some individuals or for a limited amount of time.

### Combination Therapies

The invention as described herein may also be combined with other suitable therapies or surgical procedures.

The methods and uses for treating a disorder of the left ventricle according to the present invention may be performed in combination with additional therapies, for example therapies or treatments used for treating or preventing disorders of the left ventricle including e.g. heart failure.

Such therapies may include e.g. lifestyle factors such as weight management, reducing smoking etc.

Such therapies may also include two or more medications to treat or improve left ventricle function in patients suffering from left ventricle disease such as heart failure. Such therapies may include blood pressure medication such as: angiotensin-converting enzyme inhibitors including benazepril, captopril, enalapril, fosinopril, lisinopril, moexipril, perindopril, quinapril, ramipril, trandolapril (Lotensin, Vasotec, Prinivil, Accupril, Mavik, and others) or angiotensin receptor blockers (ARBs) including azilsartan, candesartan, eprosartan, irbesartan, losartan, olmesartan, telmisartan, valsartan (Atacand, Avapro, Benicar, Diovan, Edarbi, Micardis, Teveten, and others).

It may also include combined blood pressure medication such as entresto (sacubitril/valsartan) typical used in the treatment of heart failure. Such therapies may also include beta blockers, which are a class of drugs that slows the heart rate, reduces blood pressure, limits or reverses some of the damage to the heart and reduces the risk of some abnormal heart rhythms, e.g. carvedilol (Coreg), metoprolol (Lopressor) and bisoprolol (Zebeta).

Such therapies may also include calcium channel blockers, which are medications to prevent calcium from entering cells of the heart and blood vessel walls, resulting in lower blood pressure; e.g. amlodipine (Norvasc) and diltiazem (Cardizem, Tiazac).

Such therapies may also include diuretics combined with potassium and magnesium supplements; diuretics such as fluoremide (Lasix) can help decrease fluid in the lungs and make breading easier for patients suffering from left ventricle disorders.

Such therapies may also include aldosterone antagonists, including spironolactone (Aldactone) and eplerenone (Inspra), which are potassium-sparing diuretics, and have additional properties that may help people with severe systolic heart failure live longer.

Such therapies may also include inotropes, which are intravenous medications used in people with severe heart failure in the hospital to improve heart pumping function and maintain blood pressure. Such therapies may also include digoxin (Lanoxin), a drug, also referred to as digitalis, which increases the strength of the heart muscle contractions and which also tends to slow the heartbeat. Such therapies may also include nitrates to alleviate chest pain, a statin to lower cholesterol or blood-thinning medications.

Such therapies may also include surgery to treat the underlying problem that led to left ventricular disease, e.g. aortic valve stent insertions, coronary bypass surgery or heart valve repair or replacement.

Such therapies may also include the implantation of medical devices such as an implantable cardioverter-defibrillator (ICD) to monitor and pace the heart rhythm or a biventricular pacemaker to provide cardiac resynchronization therapy (CRT).

### Composition

The population of cells according to the invention as described herein may be provided in the form of a composition.

The compositions may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Compositions, according to the current invention, are administered using any amount and by any route of administration effective for preventing or treating a subject. An effective amount refers to a sufficient amount of the composition to beneficially prevent or ameliorate the symptoms of the disease or condition.

The exact dosage is chosen by the individual physician in view of the subject to be treated. Dosage and administration are adjusted to provide sufficient levels of the active agent(s) or to maintain the desired effect in a subject. Additional factors which may be taken into account include the severity of the disease state, e.g., liver function, cancer progression, and/or intermediate or advanced stage of macular degeneration; age; weight; gender; diet, time; frequency of administration; route of administration; drug combinations; reaction sensitivities; level of immunosuppression; and tolerance/response to therapy. Long acting pharmaceutical compositions are administered, for example, hourly, twice hourly, every three to four hours, daily, twice daily, every three to four days, every week, once every two weeks, biyearly, once a year, or even as a single dose, depending on half-life and clearance rate of the particular composition.

The active agents of the pharmaceutical compositions of embodiments of the invention are preferably formulated in dosage unit form for ease of administration and uniformity of dosage. The expression “dosage unit form” as used herein refers to a physically discrete unit of active agent appropriate for the subject to be treated. The total daily, weekly, monthly, yearly or single dose usage of the compositions of the present invention will be decided by the attending physician within the scope of sound medical judgment. For any active agent, the therapeutically effective dose is estimated initially either in cell culture assays or in animal models, potentially mice, pigs, goats, rabbits, sheep, primates, monkeys, dogs, camels, or high value animals. The cell-based, animal, and in vivo models provided herein are also used to achieve a desirable concentration, total dosing range, and route of administration. Such information is used to determine useful doses and routes for administration in humans. A therapeutically effective dose refers to that amount of active agent that ameliorates the symptoms or condition or prevents progression of the disease or condition. Therapeutic efficacy and toxicity of active agents are determined by standard pharmaceutical procedures in cell cultures or experimental animals, e.g., ED₅₀ (dose therapeutically effective in 50% of the population) and LD₅₀ (dose lethal to 50% of the population). The dose ratio of toxic to therapeutic effects is the therapeutic index, which is expressed as the ratio, LD₅₀/ED₅₀. Pharmaceutical compositions having large therapeutic indices are preferred. The data obtained from cell culture assays and animal studies are used in formulating a range of dosage for human use.

As formulated with an appropriate pharmaceutically acceptable carrier in a desired dosage, the pharmaceutical composition or methods provided herein is administered to humans and other mammals for example surgically (for example via injection to the heart), depending on preventive or therapeutic objectives and the severity and nature of the disorder.

Injections of the pharmaceutical composition include for example injection directly into the heart, intravenous, subcutaneous, intra-muscular, or intraperitoneal.

Liquid dosage forms are, for example, but not limited to, intravenous, ocular, mucosal, pharmaceutically acceptable emulsions, microemulsions, solutions, suspensions, syrups, and elixirs. In addition to at least one active agent, the liquid dosage forms potentially contain inert diluents commonly used in the art such as, for example, water or other solvents. Besides inert diluents, the ocular, oral, or other systemically-delivered compositions also include adjuvants such as wetting agents, emulsifying agents, and suspending agents.

The active agent may be admixed under sterile conditions with a pharmaceutically acceptable carrier. Preservatives or buffers may be required. Administration is in a therapeutic or prophylactic form. Certain embodiments of the invention herein may be combined with implantation devices, (e.g., a pacemaker), and methods of making or using such devices or products.

In one aspect the pharmaceutical composition may be in the form of a patch, for example a patch that may be directly applied to the left ventricle. Suitable patches will be known to one skilled in the art.

In one aspect the cells may be administered in combination with other elements, for example supporting cells, carriers, loaded vesicles, microRNAs, growth factors and/or small molecules that may be advantageous for cell growth and development.

Patches have the added advantage of providing controlled delivery of the active ingredients. Such dosage forms can be made by dissolving or dispensing the compound in the proper medium. Absorption enhancers are used to increase the flux of the compound across the cells, including thick epithelium like the epicardium. Rate is controlled by either providing a rate controlling membrane or by dispersing the compound in a polymer matrix or gel.

Injectable preparations of the pharmaceutical composition, for example, sterile injectable aqueous or oleaginous suspensions are formulated according to the known art using suitable dispersing agents, wetting agents, and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or a suspending medium. For this purpose, bland fixed oil including synthetic mono-glycerides or di-glycerides is used. In addition, fatty acids such as oleic acid are used in the preparation of injectables. The injectable formulations are sterilized prior to use, for example, by filtration through a bacterial-retaining filter, by irradiation, or by incorporating sterilizing agents in the form of sterile solid compositions, which are dissolved or dispersed in sterile water or other sterile injectable medium. Slowing absorption of the agent from subcutaneous or intratumoral injection was observed to prolong the effect of an active agent. Delayed absorption of a parenterally administered active agent is accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release is controlled. Examples of other biodegradable polymers include poly (orthoesters) and poly(anhydrides). Depot injectable formulations are also prepared by entrapping the agent in liposomes or microemulsions that are compatible with body tissues.

Solid dosage forms for oral administration include capsules, tablets, pills, powders, and granules. In solid dosage forms, the active agent is mixed with at least one inert, pharmaceutically acceptable excipient or carrier such as sodium citrate, dicalcium phosphate, fillers, and/or extenders such as starches, sucrose, glucose, mannitol, and silicic acid; binders such as carboxymethylcellulose, alginates, gelatin, polyvinylpyrrolidinone, sucrose, and acacia; humectants such as glycerol; disintegrating agents such as agar-agar, calcium carbonate, potato or tapioca starch, alginic acid, certain silicates, and sodium carbonate; solution retarding agents such as paraffin; absorption accelerators such as quaternary ammonium compounds; wetting agents, for example, cetyl alcohol and glycerol monostearate; absorbents such as kaolin and bentonite clay; and lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using excipients such as milk sugar as well as high molecular weight PEG and the like. The solid dosage forms of tablets, dragees, capsules, pills, and granules are prepared with coatings and shells such as enteric coatings, release controlling coatings, and other coatings known in the art of pharmaceutical formulating. In these solid dosage forms, the active agent(s) are admixed with at least one inert diluent such as sucrose or starch. Such dosage forms also include, as is standard practice, additional substances other than inert diluents, e.g., tableting lubricants and other tableting aids such as magnesium stearate and microcrystalline cellulose. In the case of capsules, tablets and pills, the dosage forms may also include buffering agents. The composition optionally contains opacifying agents that release the active agent(s) only, preferably in a certain part of the intestinal tract, and optionally in a delayed manner. Examples of embedding compositions include polymeric substances and waxes.

### Engineered Heart Tissue

The cells according to the invention as described herein may be used in the context of an engineered heart tissue, for example to generate a cardiac patch. The present invention encompasses engineered heart tissue comprising the cells according to the invention as described herein.

In general, tissue engineering is a branch of engineering science that focuses on developing living tissue matrices under laboratory conditions. These tissue matrices may consist of a scaffold containing cells that can be used as a model systems for drug testing or applied as a graft or patch in the body to repair injured tissues or organs. Cardiac tissue engineering aims to manipulate the microenvironment that cells interact with, to facilitate cell assembly and build functional tissue. Its main goal is to provide a functional human cardiac muscle for drug discovery, studies of cardiac pathophysiology, and ultimately for cell therapy by repairing the diseased or damaged myocardium.

In vitro approaches have used different scaffolds as well as different cell types or a combination of cell types. Human pluripotent stem cell-derived cardiomyocytes have been used in this context, and are hoped to be the future of cardiac regenerative medicine. The aim is to organise human pluripotent stem cell-derived cardiomyocytes into a functional tissue that is large enough for the intended use and capable of generating a contraction force (≥2-4 mN/mm2) and propagating electric signals (at conduction velocities of ≥25 cm/s). Cells may be cultivated on a scaffold (structural and logistic template for tissue formation) in tissue culture wells or in a bioreactor (a bulk culture system providing conditions designed to achieve a desired degree of functionality).

Engineered heart tissues may be derived by experimental manipulation of pluripotent stem cells, such as embryonic stem cells (hESCs) and induced pluripotent stem cells (hiPSCs) to differentiate into human cardiomyocytes. Interest in these bioengineered heart tissues has risen due to their potential use in cardiovascular research and clinical therapies. These tissues provide a unique in vitro model to study cardiac physiology with a species-specific advantage over cultured animal cells in experimental studies. Engineered heart tissues also have therapeutic potential for in vivo regeneration of heart muscle. Engineered heart tissues provide a valuable resource to reproduce the normal development of human heart tissue, understand the development of human cardiovascular disease (CVD), and may lead to engineered tissue-based therapies for CVD patients.

The exact size of the engineered heart tissue is chosen by the individual physician in view of the subject to be treated. The cell number required to engineer such patch will be adjusted to provide enough contraction force, appropriate conduction and/or coverage, or to maintain the desired effect in a subject. Additional factors which may be taken into account include the severity of the disease state, e.g., liver function, cancer progression, and/or intermediate or advanced stage of macular degeneration; age; weight; gender; diet, time; frequency of administration; route of administration; drug combinations; reaction sensitivities; level of immunosuppression; and tolerance/response to therapy.

Administration of the engineered heart tissue may be in line with administration described herein.

The active agent may be admixed under sterile conditions with a pharmaceutically acceptable carrier. Preservatives or buffers may be required. Administration is in a therapeutic or prophylactic form. Certain embodiments of the invention herein may be combined with implantation devices, (e.g., a pacemaker), and methods of making or using such devices or products.

Engineered heart tissue, for example in the form of a patch, may be administered, for example, every month, biyearly, once a year, once every two years, or even as a single dose, depending on the patch survival and disease progression. Repeated patch administration may be required for example, despite of patch survival and disease stabilization, because the desired effect in a patient requires multiple administrations.

A therapeutically effective dose refers to engineered heart tissue size and cell content within it, which is sufficient to ameliorate the symptoms or condition or prevents progression of the disease or condition. Therapeutic efficacy and toxicity of active agents are determined by standard pharmaceutical procedures in cell cultures or experimental animals, e.g., ED₅₀ (dose therapeutically effective in 50% of the population) and LD₅₀ (dose lethal to 50% of the population). Therapeutic efficacy will be measured for example by assessing heart function and patient fitness. The dose ratio of toxic to therapeutic effects is the therapeutic index, which is expressed as the ratio, LD₅₀/ED₅₀.

Pharmaceutical compositions having large therapeutic indices are preferred. The data obtained from cell culture assays and animal studies are used in formulating a range of dosage for human use.

The present invention encompasses engineered heart tissue comprising the cells according to the invention as described herein.

Engineered heart tissues may be generated using methods described in the art (e.g. Zimmermann et al., 2004; Hansen et al., 2010), for example using variations to these methods or new methods.

Engineered heart tissues may be constructed (1) from one or more sheets of cells that are grown in monolayers and released, intact, from the culture surface, (2) by seeding the cells into the extracellular matrix of decellularized tissues, e.g. decellularized myocardial tissue, or (3) by suspending cells in a scaffold. The scaffold itself may be generated by 3D printing, which would be particularly useful to pre-pattern vascular channels within the scaffold. The engineered heart tissue may also require the addition of non-cardiomyocyte cells, including, but not exclusively, endothelial cells, endocardial cells and fibroblast cells.

As discussed herein, engineered heart tissues may comprise cells (one or more subtypes) grown in or seeded in a scaffold.

In one aspect of the invention, the cells according to the present invention may be combined with a scaffold. The invention provides an engineered heart tissue comprising cells according to the present invention as described herein and a scaffold. The invention also provides a scaffold seeded with cells according to the present invention.

Suitable scaffolds are commercially available and will be known to one of skill in the art. Scaffolds may be natural or synthetic. Suitable scaffolds may comprise materials such as collagen, fibrin, cellulose, polyglycolic acid, silk fibroin, hyaluronic acid, alginate, chitosan, heparin, gelatin methacryloyl and/or polyelectrolyte complexes. It can comprise priezoelectric polymers or piezoceramics, which are electroactive scaffolds used in tissue repair and regeneration. These types of scaffolds can deliver variable electrical stimulus without an external power source and thus favour electrical signal propagation between cardiac cells the engineered heart tissue construct. For nanofibrous scaffolds, the fibre diameters can be controlled via electrospinning, a technique that enables manufacturing of scaffolds with mechanical properties that closely mimic the native extracellular matrix. Electrospun scaffolds have porous architectures with a high surface area to volume ratio, to promote cell adhesion and migration.

In one aspect the scaffold and/or cells according to the invention may be supplemented with growth/differentiation factors, small molecules, microRNAs or vesicles such as exosomes.

In one aspect the engineered heart tissue generated with cells according to the present invention may include non-cardiomyocyte cells such as endothelial cells, endocardial cells and/or fibroblasts. In one aspect the invention provides an engineered heart tissue, preferably comprising a scaffold and cells according to the present invention as described herein, and optionally additional support cells e.g. endothelial cells, endocardial cells and/or fibroblasts. The cells may be added at specific concentrations which may be determined by one skilled in the art.

In one aspect, suitable scaffolds may be selected from the group consisting of those made using polymers; extra cellular matrix; manufactured, synthesized, or harvested from an animal donor; extra cellular matrix/polymer hybrids; natural extra cellular matrix; or native tissue constructs. The scaffolds may be designed to attract cells, such as endothelial cells, for repopulation or seeding. As such, the scaffold may comprise living cells, i.e. wherein the scaffold has been repopulated with tissue appropriate cells, such as those according to the invention as described herein, plus or minus additional support cells.

The engineered heart tissue may be in the form of a cardiac patch. Suitable methods to generate cardiac patches will be known to one of skill in the art.

In one aspect the engineered heart tissue or patch may comprise fibrin. The invention provides a patch, such as a fibrin patch, comprising a population of cells according to the invention as described herein.

Fibrin (also called Factor Ia) is a fibrous, non-globular protein involved in the clotting of blood. It is formed by the action of the protease thrombin on fibrinogen, which causes it to polymerize. The polymerized fibrin, together with platelets, forms a hemostatic plug or clot over a wound site. Fibrin may be used as the basis for a patch as described herein.

In one aspect the scaffold or patch may comprise collagen/matrigel. The invention provides a patch, such as a collagen/matrigel patch, comprising a population of cells according to the invention as described herein.

Collagen is the main structural protein in the extracellular matrix in the various connective tissues in the body. Over 90% of the collagen in the human body is type I collagen. For example, collagen type I for use according to the invention can be prepared from rat tails.

Matrigel is the trade name for a gelatinous protein mixture secreted by Engelbreth-Holm-Swarm (EHS) mouse sarcoma cells. Suitable sources of matrigel for use according to the invention are commercially available, for example BD Matrigel™ Basement Membrane Matrix (Becton Dickinson, cat no. 356234), a solubilized basement membrane preparation extracted from the Engelbreth-Holm-Swarm (EHS) mouse sarcoma, a tumor rich in extracellular matrix proteins to include laminin (a major component), collagen IV, heparan sulfate proteoglycans, and entactin/nidogen. BD Matrigel Matrix also contains TGF-beta, epidermal growth factor, insulin-like growth factor, fibroblast growth factor, tissue plasminogen activator, and other growth factors which occur naturally in the EHS tumor.

Collagen/matrigel may be used as the basis for a patch as described herein.

In one aspect the patch may comprise fibrinogen/Matrigel plus thrombin. Fibrinogen (factor I) is a glycoprotein complex, made in the liver, that circulates in the blood of all vertebrates. During tissue and vascular injury, it is converted enzymatically by thrombin to fibrin. Thrombin is a serine protease that plays a physiological role in regulating hemostasis and maintaining blood coagulation. Once converted from prothrombin, thrombin converts fibrinogen to fibrin.

In one aspect the scaffold or patch may comprise gelatin methacryloyl (GeIMA). The invention provides a patch, such as a gelatin methacryloyl patch, comprising a population of cells according to the invention as described herein.

GeIMA hydrogels have been widely used for various biomedical applications due to their suitable biological properties and tunable physical characteristics. Three dimensional GeIMA hydrogels closely resemble some essential properties of native extracellular matrix due to the presence of cell-attaching and matrix metalloproteinase responsive peptide motifs, which allow cells to proliferate and spread in GeIMA-based scaffolds. GeIMA may be used as the basis for a patch as described herein.

The 3D environment of engineered heart tissues enables self-organization of cell-types into an in vivo-like cardiac organization, which is more conducive of effective cell-cell communication. Engineered heart tissues also enable precise mechanical loading of the sarcomeres. The mechanical tension cells are under when incorporated into an engineered heart tissue may be an important step for maturation and these 3D tissues are amenable to physical conditioning, which has been shown to help further mature cardiomyocytes (Ronaldson-Bouchard et al., 2018; doi.org/10.1038/s41586-018-0016-3).

In one aspect the engineered heart tissues generated with cells according to the invention may be subject to physical conditioning with increasing intensity over time, i.e. subject to intensity pacing training to induce contractions, e.g. two weeks at a frequency increasing from 2 Hz to 6 Hz by 0.33 Hz per day, followed by one week at 2 Hz.

The engineered heart tissue, scaffold and/or patch according to the invention may be used in any of the methods/uses as described herein.

In one aspect the invention provides a method for treating or preventing a disorder of the left ventricle in a subject, comprising administering to said subject an engineered heart tissue, a scaffold or a patch as described herein.

In one aspect the invention provides an engineered heart tissue, a scaffold or a patch as described herein for use in the treatment or prevention of a disorder of the left ventricle.

### Kit

In one aspect the invention provides a kit comprising a population of cells as described herein, which comprises at least about 60% cells that are double positive for the markers HAND1 and MLC2v.

In one aspect the invention provides a kit comprising a population of cells as described herein and components required to generate an engineered heart tissue, e.g. scaffold components and/or support cells as described herein.

### Drug Screening

In one aspect the cell population as described herein may be used to screen for drugs that may be useful as a therapy in treating or preventing a disorder of the left ventricle.

In this regard, the cell population could be used for drug screening with the intention of identifying for example: 1) drugs which can improve LV function in patients with congenital heart diseases affecting the LV; and 2) safe drugs which are in pre-clinical trials, since many drugs are known to affect the proper function of the heart, namely the LV.

As such, in one aspect the invention provides a method for screening for a drug suitable for treating or preventing a disorder of the left ventricle, wherein said method comprises contacting a population of cells according to the present invention with a candidate drug. The method may further comprise analysing the effect of said candidate drug on said population of cells.

The invention also encompasses a method for screening for cardiotoxicity in respect of an agent, wherein said method comprises contacting a population of cells as described herein with an agent.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of aspects of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or aspects of this disclosure which can be had by reference to the specification as a whole. Accordingly, the terms defined immediately below are more fully defined by reference to the specification as a whole.

Amino acids are referred to herein using the name of the amino acid, the three letter abbreviation or the single letter abbreviation.

The term “protein”, as used herein, includes proteins, polypeptides, and peptides.

Other definitions of terms may appear throughout the specification. Before the exemplary aspects are described in more detail, it is to understand that this disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of’ as used herein are synonymous with “including”, “includes” or “containing”, “contains”, and are inclusive or open-ended and do not exclude additional, non-recited members, elements or method steps. The terms “comprising”, “comprises” and “comprised of’ also include the term “consisting of’.

The publications discussed herein are provided solely for their disclosure prior to the filing date of the present application. Nothing herein is to be construed as an admission that such publications constitute prior art to the claims appended hereto.

The invention will now be described, by way of example only, with reference to the following Examples, which are meant to serve to assist one of ordinary skill in the art in carrying out the invention and are not intended in any way to limit the scope of the invention.

### EXAMPLES

### Example 1—Studying the First Stages of Heart Development

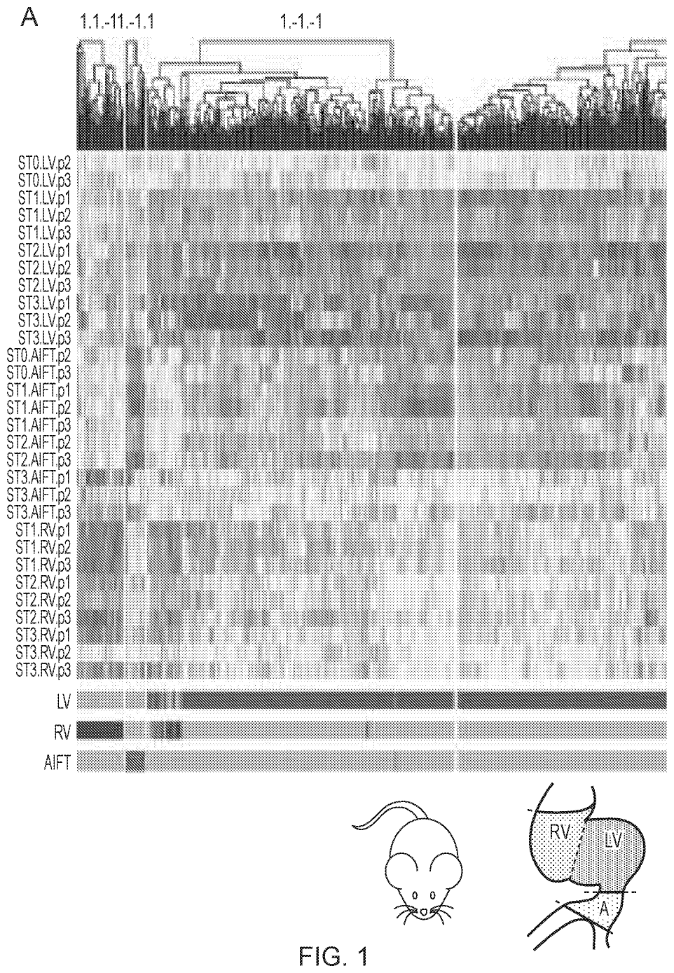

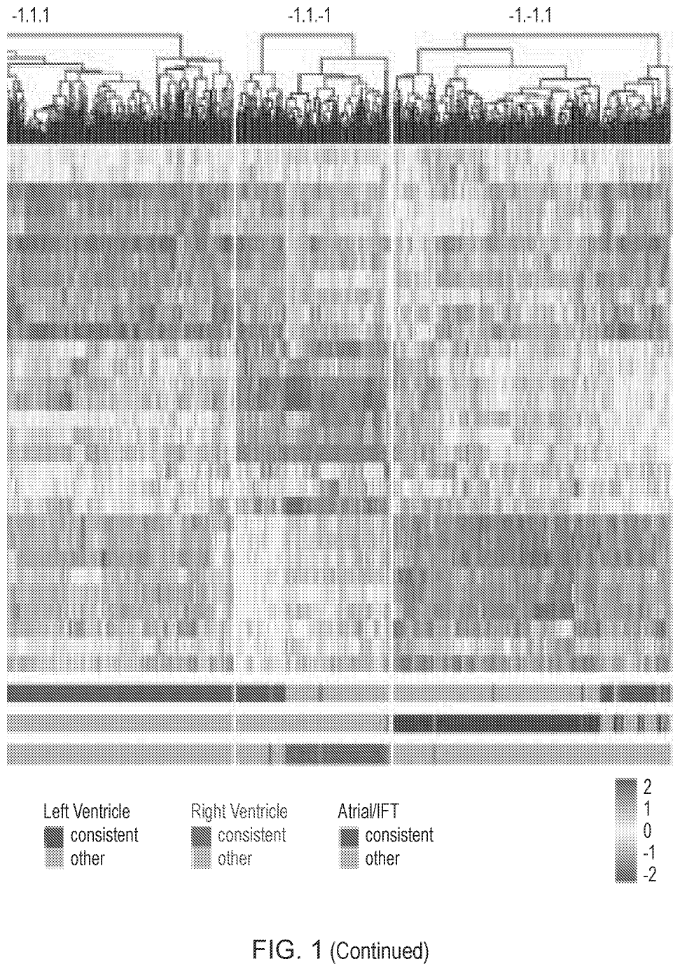

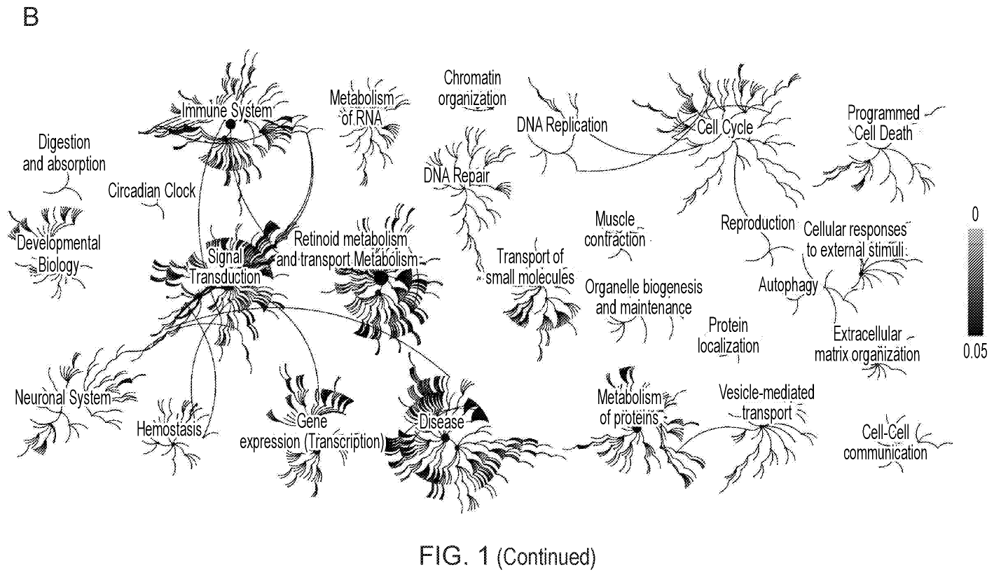

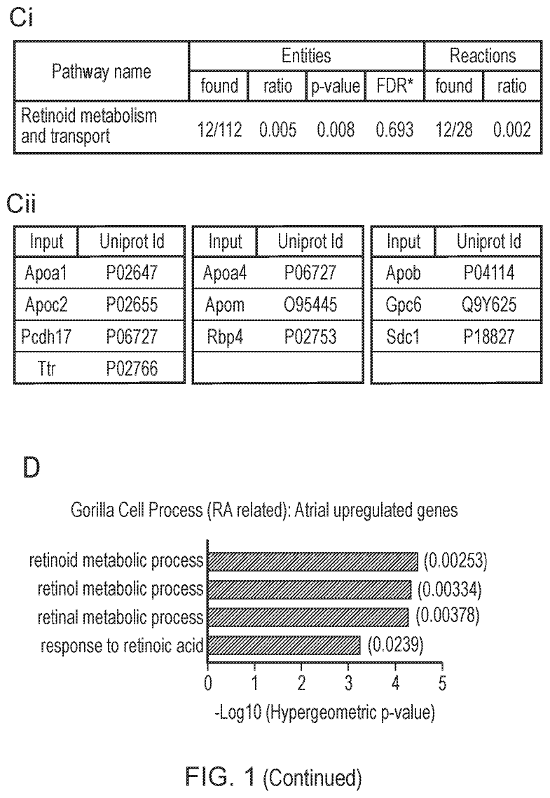

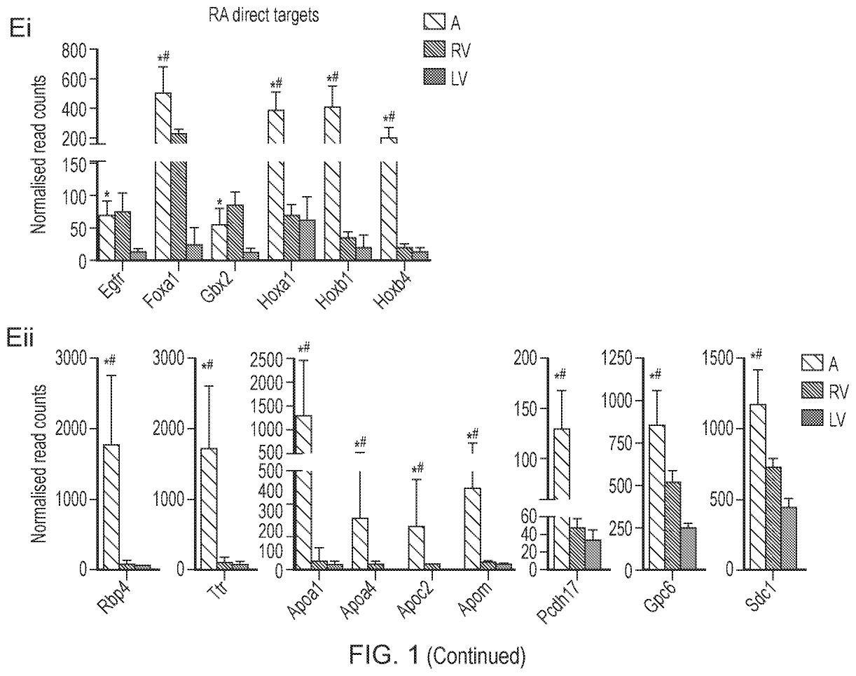

We characterized the gene expression phenotypes of different chambers of the mouse heart. Samples were analysed from microdissected hearts as exemplified on the diagram on the right of FIG. 1A, where RV corresponds to the presumptive right ventricle, LV corresponds to the presumptive left ventricle and A corresponds to the presumptive atria chamber. The stages analysed (STO-ST3) correspond to a series of stages of heart development ranging from the early heart bub (E8.25) to the end of looping (E8.5) as depicted in the diagram on the right within FIG. 1A. The genes which are consistently differentially expressed in a given region across all stages analysed, as indicated per the colour code, are highlighted in FIG. 1A.

The genes that were uniquely upregulated in atrial samples were analysed using the Reactome analysis tool. The results demonstrate that genes involved in retinoid metabolism and transport pathways were significantly enriched, constituting 16 out of the top 25 most relevant pathways (FIGS. 1B and C). The genes uniquely upregulated in atrial samples were subject to a Gorilla GO term analysis. FIG. 1D displays the results for the Retinoic acid (RA) pathway related GO terms found enriched within the gene-set.

The differential expression of retinoic acid direct target genes and genes involved in the retinoic acid pathway (as highlighted by the Reactome analysis) across the three chambers was assessed (FIG. 1E). The results represent the mean of 3 replicates at three different stages (i.e. 9 data points). Expression of retinoic acid related genes was consistently higher in the presumptive atria as opposed to the left or right ventricles.

### Example 2—Employing Lessons from the Embryo in Human Embryonic Stem Cells to Generate Left Ventricle Cardiomyocytes

Given the low expression of retinoic acid related genes within ventricle regions of the heart (FIG. 1), we hypothesised that the retinoic acid pathway needs to be off in order to generate ventricular cardiomyocytes.

In light of prior art showing that ventricular cardiomyocytes (predominantly left ventricle cardiomyocytes) are derived from a specific mid-to-anterior region of the primitive streak (Bardot et al., 2017; DOI: 10.1038/ncomms14428) we further hypothesised that we needed to emulate such streak location in vitro in order to generate the mesoderm that gives rise to ventricular cardiomyocytes, especially left ventricular cardiomyocytes.

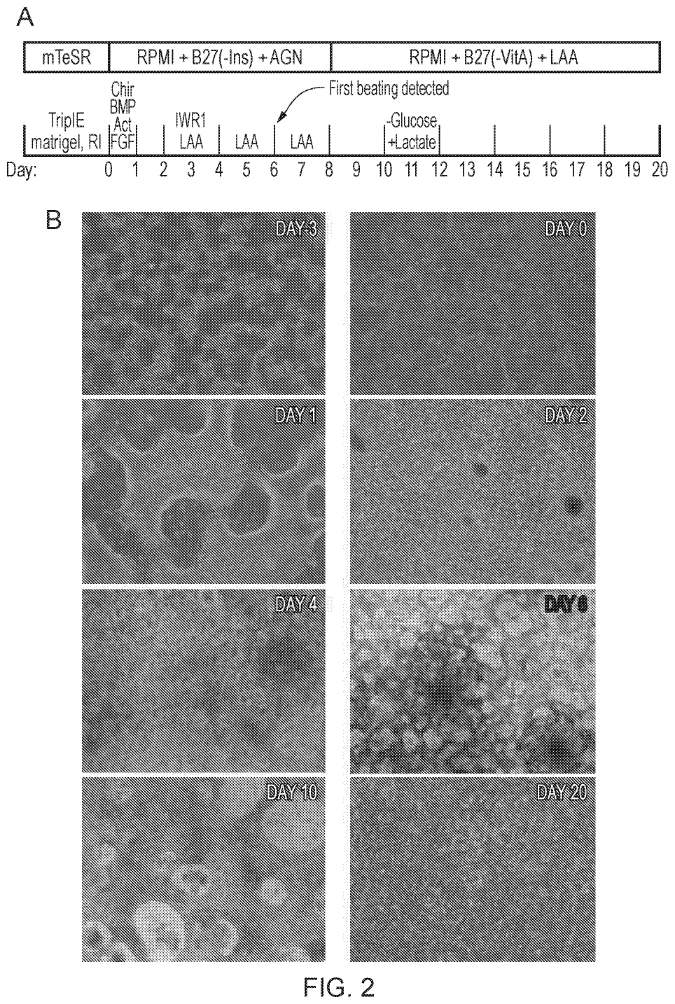

The above lead us to devise a protocol to generate left ventricle specific cardiomyocytes. We started by modulating the levels of Activin, BMP, and Wnt within the first step of the differentiation protocol aiming to generate the mesoderm with left ventricle cardiomyocyte potential (FIG. 2A). We also modulated the retinoic acid signaling pathway from day 0 of differentiation to achieve an homogeneous population of left ventricle cardiomyocytes (FIG. 2A); this was critical in the first 8 days of differentiation. Thereafter, cells had to be kept in media devoid of vitamin A or retinoic acid (FIG. 2A). A schematic of the hPSC cardiomyocyte differentiation protocol described herein can be found on FIG. 2A, where the different steps and components used in the protocol are indicated.

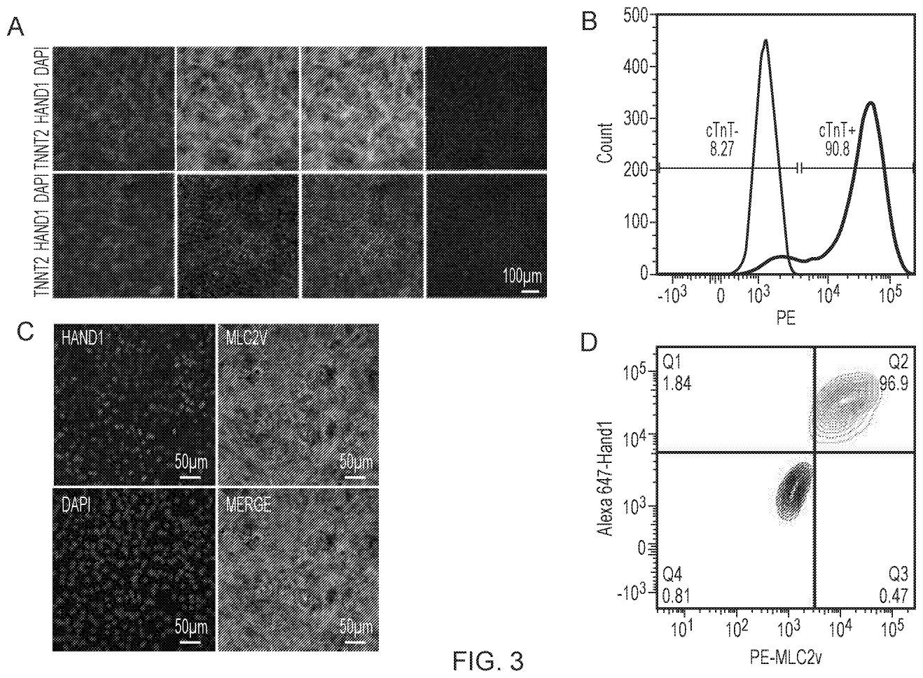

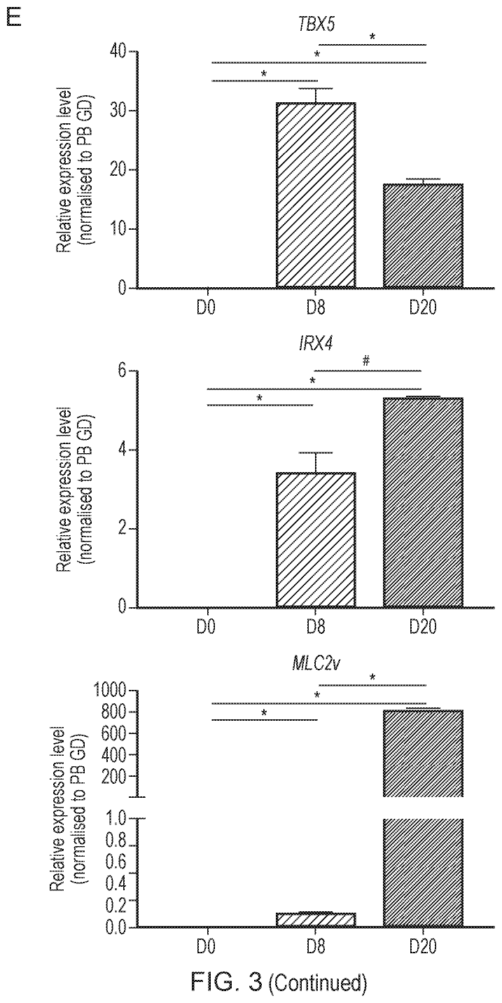

According to this protocol, cells go through specific developmental stages, i.e. appropriate mesoderm patterning followed by induction of first heart field cardiac progenitors, followed by left ventricle cardiomyocyte patterning and maturation. Cells can be further matured over time in culture. Beating is first observed at day 6 of differentiation. By day 10 cells beat fast. At day 20 we obtain a homogenous population of left ventricle cardiomyocytes, as specified by the double expression of MLC2V and HAND1, which beats slowly (FIGS. 3A, 3C, 3D). As expected cells also express the pan-cardiomyocyte marker TNNT2 (FIGS. 3A and 3B). The cells exhibit relatively organized myofibrils (FIG. 3C) and further express the pan ventricular marker IRX4 and the left ventricle and atrial marker TBX5 (FIG. 3E). Moreover cells express very high levels of MLC2v (FIG. 3E). Representative images of the various steps along the left ventricle cardiomyocyte differentiation protocol can be found on FIG. 2B.

The protocol for producing the cells is as described below:

Differentiation of Pluripotent Stem Cells

Maintenance cells were grown for 4-5 days (WA09 normally for 5 days) until colonies reached confluency. Cells were passaged around lunch time every time and on average at a split ratio of 1:10-1:20.

Setting up an experiment:
1. Split cells with TrypLE for 5 min at 37° C.
2. Quench cells with 4× the amount of media, take a small aliquot to count cells and spin down the remaining cells for 5 min at 900-1200 rpm.
3. Resuspend cells in media containing 10 μM rock inhibitor (RI).
4. Plate 0.3-0.8×10⁵ cells per cm² into wells coated for 1 h with ES cell-graded Matrigel (or growth factor reduced Matrigel) and containing media supplemented with 10 uM rock inhibitor. Let cells settle overnight.
5. On the next day feed cells with hPSC media devoid of RI.
6. Feed cells every day until they are ready to start an experiment (it can take up to 4 days). Note the cell density should be about 80% confluent at the start.
7. Cells should be pluripotent on day 0 as per co-expression of OCT4, NANOG, SOX2 and absence of SSEA1 in more than 90% of the cells.

Cardiomyocyte Differentiation Protocol:
1. On day 0 start differentiation by removing the hPSC media and adding 1 ml of heart media 1 containing Activin A (0-25 ng/ml), FGF2 (5-25 ng/ml), BMP4 (0-25 ng/ml) and Chiron (1-12 uM). Concentrations are dependent on cell density and cell line.
2. On day 1 add 2 ml of heart media 1 devoid of extra factors.
3. On day 2 add 2 ml of heart media 1 containing L-AA and IWR (1-5 uM).
4. On day 4 add 2 ml of heart media 1 containing L-AA.
5. On day 6 add 2 ml of heart media 1 containing L-AA. At this stage cells should start to beat.
6. On day 8 add 2 ml of heart media 2 containing L-AA. At this stage the entire dish should be beating rapidly and bundles of beating cells should be connected with each other.
7. On day 10 add 2 ml of heart media 3 containing L-AA. Normally cultures are relatively homogeneous for cardiomyocytes but depending on the cell line there may be more or less additional cells distinguishable on the dish—usually they look like a monolayer underneath the cardiomyocytes. Heart media 3 is designed to metabolically select the cardiomyocytes. This media can be toxic to the cells so the amount of time cells are exposed to it can vary from cell line to cell line or even between experiments.
8. Feed cells every other day with 2 ml of heart media 3 containing L-AA until mostly cardiomyocytes remain the cultures. If cells are showing signs of struggle in the metabolic selection media (heart media 3) remove them immediately and feed cells with heart media 2 instead. Note: best results, i.e. more homogeneous populations, can be achieved if cells are exposed to at least two days of metabolic selection. Normally, cells are kept in heart media 3 for 2 days only, and definitely never longer than 4-6 days.
9. On day 15 split cells into a new dish coated with growth factor reduced Matrigel. Use the STEMdiff cardiomyocyte dissociation kit (Stem cell technologies, #05025) to split the cells and replate them in the provided support medium supplemented with 10 μM rock inhibitor. NOTE: Some cell lines may need more or less cells replated, the optimum is to have enough cells on the dish to generate a network of cardiomyocytes beating as a monolayer.
10. From day 16 feed cells every other day with heart media 2 containing L-AA.
11. Cells will continue to mature on the dish and will be ready for analysis around day 20.

Heart Media 1:
1. RPMI 1640 medium (11875, Life Technologies) containing glucose and L-glutamine.
2. B27 supplement minus insulin (1 ml per 50 ml RPMI).
3. AGN193109 (final concentration of 20-200 nM).

Heart Media 2:
1. RPMI 1640 medium (11875, Life Technologies) containing glucose and L-glutamine.
2. B27 supplement plus insulin minus vitamin A (1 ml per 50 ml RPMI).
3. OPTIONAL: AGN (final concentration of 20-200 nM).

Heart Media 3:
1. RPMI 1640 medium (11879, Life Technologies) containing L-glutamine but NO glucose.
2. B27 supplement plus insulin minus vitamin A (1 ml per 50 ml RPMI).
3. OPTIONAL: AGN (final concentration of 20-200 nM).
4. 4 mM L-lactic acid

### Example 3—Functional Characterization of Left Ventricular Cardiomyocytes

We characterized the functionality of cardiomyocytes produced using the left ventricle cardiomyocyte protocol by looking at the electrophysiology properties of the cells as well as at the calcium transients.

The local extracellular action potential (LEAP) signal of the cardiomyocytes was acquired using the Axion Biosystems MEA system. The day 20 cardiomyocytes exhibit a ventricular action potential shape, i.e. a plateau followed by a sharp repolarization phase (FIG. 4A). This result was corroborated using the Maxwell Biosystems MEA system (data not shown) and by using optical mapping (CellOptic, data not shown). Furthermore, principal component analysis of the field potentials determined using the Maxwell Biosystems MEA system revealed that only one population of cells could be identified (FIGS. 4Bi and 4Bii). The average beat rate (bpm) of day 20 cardiomyocytes was determined using di-4-ANEPPS and optical mapping (CellOptic). Even as early as day 20 of differentiation the beat rate for these cardiomyocytes is slow, a sign the cardiomyocytes are becoming mature in early cultures (FIG. 4C). Furthermore, the cardiomyocytes demonstrated a regular and uniform periodicity of beating (FIG. 4D).

A conduction velocity analysis demonstrated that LV-like cardiomyocytes have a conduction velocity close to that of neonatal cardiomyocytes (0.3 m/s; FIG. 4E). This offers an improvement over the prior art, which has achieved conduction velocities of between 0.035 m/s at day 18 to 0.12 m/s on day 28 (Zhu et al., 2017; Scientific Reports, 7:43210).

A beat amplitude mean analysis demonstrates LV-like cardiomyocytes have stronger contraction force than commercially available cardiomyocytes (FIG. 4F). We further showed via excitation-contraction delay analysis that LV-like cardiomyocytes take longer to contract post action potential initiation than commercially available cardiomyocytes (FIG. 4G), likely because they exhibit a longer plateau associated with the opening/closing of slow calcium channels, typical of ventricular cardiomyocytes (FIG. 4A).

A field potential duration analysis revealed the LV-like cardiomyocytes demonstrate an FPD range between 300 ms-500 ms (FIG. 4H), which was on average longer than what has been described in the literature for hPSC-derived cardiomyocytes generated using protocols known in the art, and approximately the length of a typical ventricular action potential (Coppini et al., 2014; doi:10.3791/51116). Additionally, these day 20 cardiomyocytes have a fast firing ability, as judged by action potential rise time (Trise) analysis (FIG. 4I).

The action potential duration at 30% (ADP30), 50% (ADP50), and 90% (ADP90) depolarisation, and action potential triangulation as determined by the ratio between ADP50 and APD90 are consistent with what has been described for ventricular cells (FIG. 4J). Specifically, they highlight that ADP50 and ADP90 are not very far apart in keeping with the existence of a plateau followed by a rapid repolarization, which are key features of the ventricular action potential shape. This is in contrast to commercially available cardiomyocytes, which have a triangular-like action potential shape, i.e. absence of a plateau (Figure A), and therefore exhibit a smaller triangulation ratio.

The average calcium transient (CaT) was determined using Fura-4F (FIGS. 4K and 4L); the results illustrate the CaT peaks towards the end of the action potential plateau (FIGS. 4A and 4K), and is followed by cell contraction (FIG. 4G), demonstrating the cells respond appropriately to calcium, i.e. they exhibit excitation-contraction coupling. CaT are close to those seen in human adult ventricular cardiomyocytes.

### Example 4—Subcellular Characterization of Left Ventricular Cardiomyocytes

We further characterised the subcellular structure of cardiomyocytes produced using the left ventricle cardiomyocyte protocol.

Transmission electron microscopy (TEM) was used to analyse the ultrastructure of cardiomyocytes at day 20 of differentiation, in particular the nucleus, mitochondria, and sarcomeres (FIG. 5A). Of note is the remarkable organisation of the sarcomeres at day 20 of differentiation that can be seen in FIG. 5A (iii), something never reported for monolayer cultures prior to day 60 of differentiation. The average length of typical human adult ventricle cardiomyocytes is 1.7 μm (Nguyen et al., 2017; doi: 10.3389/fphys.2017.01073). The average sarcomere length of cardiomyocytes produced using the left ventricle cardiomyocyte protocol was approaching 1.7 μm at day 20, and by day 60 the cardiomyocytes demonstrated nearly the length of a fully mature ventricle sarcomere (FIG. 5B; grey dotted line).

The mean mitochondrial DNA copy number at days 0, 10, 20, 40 and 60 of differentiation was also characterised. There was a sharp increase between days 10 and 20, suggesting an increased mitochondrial activity from then onwards, which is in keeping with the cells having an active metabolism (FIG. 5C).

The cardiomyocytes were stained with MitoTracker, a mitochondrial marker, to determine the mitochondrial network of these cells. We performed these experiments in cells endogenously GFP tagged at the MLC2v locus to identify in live cells the thick filaments of the sarcomere, and the live nuclear stain Hoechst to identify the nuclei. Confocal microscopy analysis of these cells showed the cardiomyocytes displayed extensive interconnected mitochondrial networks (FIG. 5D), typical of neonatal cardiomyocytes (Eisner et al., 2017; doi.org/10.1073/pnas.1617288114). Next we focused on characterizing the cytoarchitecture of the day 20 left ventricle cardiomyocytes. To this end, cardiomyocytes were stained with a panel of antibodies and analysed by confocal microscopy. In keeping with the TEM results, cells show a well-developed myofibrillar arrangement already at day 20 of differentiation, as shown by staining for the Z-disc protein SARCOMERIC ALPHA-ACTININ (FIG. 6A), with relatively little presence of pre-myofibrils, which are the earliest stage of myofibril assembly in cultured cardiomyocytes (Rhee et al., 1994; doi: 10.1002/cm.970280102). Myofibril alignment was also assessed by staining for the intermediate filament protein DESMIN, which in mature cardiomyocytes shows cross-striations at the Z-disc level compared to filamentous arrays stretching throughout the cytoplasm in immature cells (Ehler et al, 1999; PMID:10212147; Kim 1996; PMID: 8888968). While not as perfect as in the mature heart, DESMIN striations are also already evident in day 20 cardiomyocytes, in addition to DESMIN filamentous signals and concentration at cell-cell contacts (FIG. 6D).

We also analysed the cell-cell communication maturity of the cells by looking at CONNEXIN-43, the major gap junction protein in the adult cardiomyocyte (Hirschy et al., 2006; doi: 10.1016/j.ydbio.2005.10.046) and using confocal microscopy. In day 20 cardiomyocytes, occasional punctate signal could be detected; the location of these puncta at the cell-cell contacts between the cardiomyocytes and the signal intensity confirm that these are indeed gap junctions (FIG. 6A).

Lastly, we analysed sarcomere maturity by looking at mature markers of both the Z-disk and the M-band and using confocal microscopy.

TELETHONIN (also known as T-cap for titin cap) provides an extremely tight connection between the N-termini of titin in the Z-disc in the form of a sandwich-like structure (Zou et al., 2006; doi: 10.1038/nature04343). In the adult cardiomyocyte TELETHONIN is absent from the transitional junction, which is where new sarcomeres can get inserted during stress to cope with increasing demands. TELETHONIN expression is also only upregulated during later embryonic development and in primary neonatal cultures of rat cardiomyocytes, depending on rat strain and exact age of the pups, between one and two third of the cardiomyocytes are still TELETHONIN negative. In the day 20 cardiomyocyte cultures, a subset of cells do express TELETHONIN in the Z-disc, yellow signal due to the overlay with the Z disc marker SARCOMERIC ALPHA-ACTININ (FIG. 6B). TELETHONIN has not previously been observed in monolayer cultures of cardiomyocytes produced using methods described in the art and was reported to be present in only 9% of the cardiomyocytes available commercially via Axol (Zuppinger et al., 2017; doi:10.4081/ejh.2017.2763), thus confirming the method described herein generates more mature cardiomyocytes.

The composition of the M-band, a structure in the middle of the sarcomere that links the thick (myosin and associated proteins) with the elastic filament system composed of titin, changes depending on the developmental status (Lange et al., 2020; doi: 10.1016/j.bbamcr.2019.02.003). MYOMESIN is constitutively expressed as the major crosslinker between myosin and titin, but around birth, the upregulation of M-PROTEIN expression marks the mature status of M-bands in the ventricle. A subset of the day 20 cardiomyocytes is positive for M-PROTEIN similar to what is observed in rat neonatal cardiomyocytes (FIG. 6C). M-PROTEIN has been previously seen in monolayer cultures of cardiomyocytes produced using methods described in the art but never as early as day 20 of differentiation (Kamakura et al., 2013; 10.1253/circj.cj-12-0987; Fleischer et al., 2019;

doi.org/10.1016/j.bios.2018.10.061).

The above results demonstrate that the cardiomyocytes produced using the left ventricle cardiomyocyte protocol are both functionally and phenotypically reaching full maturity.

### Example 5—Generation and Characterization of Engineered Heart Tissues Generated Using Day 40 Cardiomyocytes

Engineered heart tissues (EHTs) were generated following the protocol developed by the Eschenhagen lab (Hansen et al., 2010; 10.1161/CIRCRESAHA.109.211458).

We characterized the EHTs generated using day 40 cardiomyocytes produced using the left ventricle cardiomyocyte protocol (LV-EHTs). Remarkably, the LV-EHTs started beating at day 9, though the spontaneous beat rate was low and remained low or inexistent thereafter (FIG. 7B). In other experiments, beating was never observed (data not shown). Importantly, the LV-EHTs could be paced (FIG. 7B). This demonstrates the relatively mature ventricular phenotype of the in vitro derived left ventricle cardiomyocytes, since adult ventricular cardiomyocytes only beat when stimulated, and the LV-EHTs could be paced even while unable to spontaneously beat. The properties of the LV-EHTs while spontaneously beating were compared with those of EHTs generated with other hPSC-derived cardiomyocytes (FIG. 7C-F). The results confirmed the unique and more mature slow beat rate of the LV-EHTs. The LV-EHTs generated an average amount of force, and displayed average times to reach 20% contraction and 20% relaxation in comparison to other EHTs tested.

In conclusion, the above results show in vitro derived left ventricle cardiomyocytes as described here can be used to generate engineered heart tissues, which loose spontaneous pacemaker ability in line with what is expected for more mature ventricular cardiomyocytes. These can beat when elicited and produce some contraction force.

### DISCUSSION

Heart disease is the leading cause of death in industrialized countries, with myocardial infarction being the most common cause of heart injury. Upon myocardial infarction, a dramatic loss of contractile heart muscle (cardiomyocytes) occurs, generally affecting particularly the left ventricle. Post-infarction heart failure, for which the only cure is heart transplant, is a life limiting condition and a significant financial burden to hospitals. The key to improving the long-term outcome of these patients is to repopulate the left ventricle with functional cardiomyocytes exhibiting physiological properties as close as possible to those of adult left ventricle cardiomyocytes.

Human pluripotent stem cells (hPSCs) offer great hope for regenerative medicine, and indeed it is now routine in many laboratories to generate cardiomyocytes in vitro from hPSCs. However, current protocols produce heterogenous populations of atrial and ventricular cardiomyocytes, which are unsuitable for replacement therapies. Atrial and ventricular cardiomyocytes are vastly different. Moreover, while right and left ventricle cardiomyocytes are similar, they arise from different progenitors and display some structural, electrophysiological, metabolic and calcium handling differences.

To overcome the issue of heterogeneity, we used our knowledge of mammalian embryonic development to generate near homogeneous populations of left ventricle-like cardiomyocytes from hPSCs. We followed a two-step approach in which we first generated mesoderm progenitors with the ability to give rise to left ventricle cardiomyocytes. In the second step, we promoted left ventricle identity by switching off the retinoic acid pathway in keeping with the inactivated status of this pathway in mouse ventricle cardiomyocytes. Our approach generates more than 90% left ventricle-like cardiomyocytes at day 20 of differentiation as determined by co-expression of MLC2V and HAND1, and it has been validated in four different cell lines. These cells display a ventricular-like action potential shape with a long plateau followed by a fast repolarization stage (average triangulation=0.87); they have a conduction velocity close to that of neonatal cardiomyocytes; they can be paced; and they exhibit calcium transients close to those seen in human adult ventricular cardiomyocytes (average Tpeak=190 ms).

The cells at day 20 display a remarkable level of maturity for such a short differentiation time: they exhibit a well-developed myofibrillar arrangement including defined Z-disks, a sarcomere length approaching that of fully mature ventricle cardiomyocytes, extensive interconnected mitochondrial networks as seen in neonatal ventricle cardiomyocytes, and they express high levels of mitochondrial DNA. Moreover, various cells express the mature Z-disk marker TELETHONIN, the mature M-band marker M-PROTEIN and a few also express the mature gap-junction marker CONNEXIN43. Importantly, these cells slow down their rate of beating during time in culture and, when used to generate engineered heart tissues at day 40, they barely beat but can be paced. This loss of a spontaneous pacemaker is in line with what is expected for more mature ventricular cardiomyocytes.

1. A population of cells which comprises at least about 60% cells that are double positive for the markers HAND1 and MLC2v, wherein said cells are left ventricular cardiomyocytes.

2. The population of cells according to claim 1 which comprises at least about 65, 70, 75, 80, 85, 90, 95 or 100% cells that are double positive for the markers HAND1 and MLC2v.

3. The population of cells according to claim 1 or claim 2 which comprises at least about 65, 70, 75, 80, 85, 90, 95 or 100% cells that are positive for the markers TBX5, and HAND1 and MLC2V.

4. The population of cells according to any one of claims 1 to 3 which comprises at least about 85% cells which are double positive for the markers HAND1 and MLC2v.

5. The population of cells according to any one of claims 1 to 4 which comprises at least about 85% cells which are positive for the markers TBX5 and HAND1 and MLC2v.

6. The population of cells according to any one of claims 1 to 5 which comprises at least about 85% cells which are positive for the markers IRX4, TBX5 and HAND1 and MLC2v.

7. The population of cells according to any one of claims 1 to 6 which comprises mature cardiomyocytes.

8. The population of cells according to claim 7 wherein said mature cells exhibit hallmarks of maturity, preferably selected from:
(i) ventricular action potential shape;
(ii) slow beat rates and/or beat periodicity;
(iii) conduction velocity characteristic of neonatal ventricular cells;
(iv) higher beat amplitude mean;
(v) field potential duration in keeping with that of ventricular cardiomyocytes;
(vi) fast action potential rise time (Trise)
(vii) calcium transients (CaTs) close to those seen in human adult ventricular cardiomyocytes; and
(viii) improved CaT rise time (time to peak, Tpeak) and CaT.

9. The population of cells according to claim 7 or claim 8 wherein said mature cells exhibit improved markers of maturity, preferably selected from:
(i) sarcomere organisation, length and function;
(ii) display of mature gap-junction marker CONNEXIN-43, SARCOMERIC ALPHA-ACTININ, the mature Z-disk marker TELETHONIN, the mature M-band marker M-PROTEIN, and presence of the cardiomyocyte associated intermediate filament DESMIN at the Z-disk;
(iii) display of an extensive interconnected mitochondrial network typical of neonatal cardiomyocytes; and
(iv) increased activation of mitochondrial DNA.

10. The population of cells according to any one of claims 1 to 9 wherein said cells can be paced.

11. A method for treating or preventing a disorder of the left ventricle in a subject, comprising administering to said subject a population of cells which comprises at least about 60% cells that are double positive for the markers HAND1 and MLC2v according to any one of claims 1 to 10.

12. The method according to claim 11 wherein said disorder of the left ventricle is selected from myocardial infarction, heart failure, left ventricular hypertrophy, hypoplastic left heart syndrome, and left ventricular non-compaction cardiomyopathy (LVNC).

13. The method according to claim 12 wherein said disorder of the left ventricle is myocardial infarction.

14. The method according to claim 12 wherein said disorder of the left ventricle is heart failure.

15. The population of cells according to any one of claims 1 to 10 for use in the treatment or prevention of a disorder of the left ventricle.

16. The population of cells for use according to claim 15 wherein said disorder of the left ventricle is selected from myocardial infarction, heart failure, left ventricular hypertrophy, hypoplastic left heart syndrome and left ventricular non-compaction cardiomyopathy (LVNC).

17. The population of cells for use according to claim 16 wherein said disorder of the left ventricle is myocardial infarction.

18. The population of cells for use according to claim 16 wherein said disorder of the left ventricle is heart failure.

19. A method for preparing a population of cells which comprises at least about 60% cells that are double positive for the markers HAND1 and MLC2v, wherein said method comprises the step of culturing a pluripotent stem cell in a medium comprising a retinoic acid receptor antagonist or inverse agonist.

20. The method according to claim 19 wherein said method has a duration of between 15 and 25 days, preferably 20 days, wherein preferably the population of cells displays the hallmarks/markers of maturity according to claims 8 and/or claim 9.

21. The method according to claim 19 or claim 20 wherein said pluripotent stem cell is an embryonic stem cell or an induced pluripotent stem cell.

22. The method according to any one of claims 19 to 21 which comprises culturing said cell population with a glycogen synthase kinase-3 (Gsk3) inhibitor, preferably Chiron, BMP (preferably BMP4), Activin (preferably Activin A) and FGF (preferably FGF2).

23. The method according to claim 22 comprising culturing said cell population with the following amounts:
(i) about 1-6 μM/ml a glycogen synthase kinase-3 (Gsk3) inhibitor, preferably Chiron, preferably about 2-4 or 2-3 μM/ml Chiron;
(ii) about 1-10 ng/ml BMP, preferably BMP4, preferably about 1 to about 6 ng/ml, or about 3 to about 5 ng/ml BMP;
(iii) about 1-10 ng/ml Activin, preferably Activin A, preferably about 3 to about 10 ng/ml Activin, preferably about 5 ng/ml Activin;
(iv) about 1-10 ng/ml FGF, preferably FGF2, preferably about 3 to about 10 ng/ml, preferably about 5 ng/ml FGF.

24. The method according to any one of claims 19 to 23 wherein the method comprises culturing said cell population with a Wnt inhibitor.

25. The method according to any one of claims 19 to 24 wherein the method subsequently comprises culturing the cells in a medium which does not contain vitamin A.

26. The method according to any one of claims 19 to 25 wherein said method comprises the protocol set out in Table 1.

27. A population of cells obtained or obtainable by the method according to any one of claims 19 to 26.

28. The population of cells according to any one of claims 1 to 10 and claim 27 for use in the treatment or prevention of a disorder of the left ventricle.

29. A method for treating or preventing a disorder of the left ventricle in a subject, comprising administering to said subject a population of cells according to any one of claims 1 to 10 and claim 27.

30. A method for screening for a drug suitable for treating or preventing a disorder of the left ventricle, wherein said method comprises contacting a population of cells according to any one of claims 1 to 10 and 27 with a candidate drug.

31. A method for screening for cardiotoxicity in respect of an agent, wherein said method comprises contacting a population of cells according to any one of claims 1 to 10 and 27 with an agent.

32. A scaffold seeded with a population of cells according to any one of claims 1 to 10 and 27.

33. Engineered heart tissue comprising a population of cells according to any one of claims 1 to 10 and 27 or the scaffold according to claim 32.

34. A cardiac patch comprising a population of cells according to any one of claims 1 to 10 and 27 or the scaffold according to claim 32.

35. The engineered heart tissue, patch or scaffold according to any one of claims 32 to 34 which comprises any one or more of fibrinogen, fibrin, Matrigel, thrombin, collagen and gelatin methacryloyl.

36. The engineered heart tissue, patch or scaffold according to any one of claims 32 to 35 which further comprises non-cardiac cells, preferably selected from endothelial, endocardial and fibroblast cells.
